(19) United States
(12) Patent Application Publication
(10) Pub. No.: US 2023/0374068 A1
(43) Pub. Date: Nov. 23, 2023

(54) COMPOSITIONS AND METHODS FOR TREATING A DISEASE

(71) Applicant: RAMOT AT TEL AVIV UNIVERSITY LTD., Tel Aviv (IL)

(72) Inventors: Ronit SAGI-EISENBERG, Tel Aviv (IL); Jana OMAR, Tel Aviv (IL)

(21) Appl. No.: 18/223,730

(22) Filed: Jul. 19, 2023

Related U.S. Application Data
(63) Continuation of application No. PCT/IL2022/050155, filed on Feb. 6, 2022.
(60) Provisional application No. 63/148,231, filed on Feb. 11, 2021.

(51) Int. Cl. C07K 7/02; A61P 25/16

(52) U.S. Cl. CPC C07K 7/02; A61P 25/16; A61K 38/00

(57) ABSTRACT

The invention provides a polypeptide comprising five (5) or more amino acids of an amino acid sequence having at least 70% identity to the amino acid sequence as set forth in amino acid sequence ID NO:1; and five (5) or more amino acids of an amino acid sequence having at least 70% identity to amino acid sequence ID NO: 2; wherein amino acid sequence ID NO:1 and amino acid sequence ID NO: 2 refer to Interface 2 and Interface I, respectively, and are derived from human Rab 12 protein. The invention further provides a method of treating a disease, caused by imbalance of Rab 12 phosphorylation, or imbalance of Rab 12 interactions with its effectors via Interface I or Interface II or both, such as, Parkinson's disease.

### CROSS REFERENCE TO RELATED APPLICATIONS

This application is a bypass continuation of PCT Patent Application No. PCT/IL2022/050155 having International filing date of Feb. 6, 2022, which claims the benefit of priority of U.S. Provisional Patent Application No. 63/148,231, filed Feb. 11, 2021, the contents of which are all incorporated herein by reference in their entirety.

### REFERENCE TO AN ELECTRONIC SEQUENCE LISTING

The contents of the electronic sequence listing (RMT-008-PCT.xml; Size: 42,372 bytes; and Date of Creation: Jul. 18, 2023) is herein incorporated by reference in its entirety.

### BACKGROUND OF THE INVENTION

The small GTPase Rab12 controls biosynthetic functions such as endocytic transport and autophagy, and regulated functions, such as negative control of mast cell (MC) exocytosis, whereby the latter function is mediated by promoting retrograde transport of the MC secretory granules (SGs). A screen of Rab GTPases for their functional and phenotypic impact on MC exocytosis has identified 30 Rabs as potential regulators of this process. Among these Rabs, a constitutively active mutant of Rab12 was found to inhibit exocytosis by stimulating microtubule dependent retrograde transport of the MC SGs, promoting their perinuclear clustering. Rab12 is one of the less characterized Rabs. Previous studies have implicated Rab12 in controlling transport of specific cargo, such as the transferrin receptor, from the endocytic recycling compartment (ERC) to lysosomes and stimulating autophagy by regulating the transport of the amino acid transporter PAT4. Further studies implicated Rab12 in autophagosome trafficking and retrograde transport of the Shiga toxin. However, the underlying mechanisms of the diverse functions of Rab12 remain poorly understood. Rab GTPases perform their functions by the recruitment of effector proteins that bind to their active, GTP-bound conformation. The latter include motor proteins, SNAREs, tethering factors, cytoskeleton and cargo proteins, whose recruitment allow Rabs to regulate distinct steps along vesicular trafficking.

There are direct evidences showing that Rab12 is involved in Musician's and other Dystonias: Rab12 mutations were found in musician's dystonia (MD) and writer's dystonia (WD), which are task-specific movement disorders. Rab12 variants were not identified in healthy controls. Further Rab12 is involved in retinal ganglion cell death-associated with glaucoma. Further, there are indirect evidences showing that Rab12 is involved in Amyotrophic lateral sclerosis (ALS) because Rab12 is known to interact with OPTN/optineurin, mutations in which are associated with ALS. Moreover, there are evidences showing that Rab12 is involved in Parkinson's disease (PD). Rab12 is a physiological substrate of LRRK2, mutations in which comprise the most common cause of familial PD. LRRK2 has been implicated in inflammatory diseases including: leprosy, tuberculosis and inflammatory bowel diseases. For example, GWAS has identified LRRK2 as a major susceptibility gene for Crohn's disease.

There is a need thus to develop a medicament that will regulate Rab12 impaired interactions with its effector proteins for treating diseases in which Rab12 function is impaired.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A, 1B, 1C and 1D show that RILP, RILP-L1 and RILP-L2 form homocomplexes, but neither protein can form heterocomplexes.

FIGS. 1A, 1B show the results of immunoprecipitations in which cell lysates derived from the rat mast cell line RBL-2H3, herein referred to as RBL cells, that were co-transfected with 17.5 μg of pEGFP plasmid encoding either RILP, RILP-L1 or RILP-L2 and 17.5 μg of pEF plasmid encoding either T7-RILP, T7-RILP-L1 or T7-RILP-L2, as indicated, were subjected to immunoprecipitation with rabbit polyclonal antibodies directed against GFP. FIGS. 1C, 1D show the results of immunoprecipitations in which the RBL cell lysates described above were subjected to immunoprecipitation with mouse monoclonal antibodies directed against the T7 epitope. Immune complexes were then analyzed by SDS-PAGE and immunoblotting with mouse monoclonal anti T7 antibodies, followed by reprobing with polyclonal anti GFP antibodies (FIGS. 1A, 1B), or with polyclonal anti GFP antibodies followed by reprobing with monoclonal anti T7 antibodies (FIGS. 1C, 1D), as indicated. Input=10% of total protein.

FIGS. 2A, 2B and 2C present the results of mapping Rab12 binding sites for RILP family effectors.

FIG. 2A presents a proposed consensus sequence, based on sequence similarity of the regions neighbouring the lysine residues that are important for RILP binding to mouse Rab7 and Rab34 (boxed). FIG. 2B presents the results of pulldown experiments, in which cell lysates (500 μg) derived from RBL cells that were transiently transfected with 35 μg of either pEF-T7-RILP, pEF-T7-RILP-L1, or pEF-T7-RILP-L2, were incubated for 18 h at 4° C. with 20 μg of GST, or GST-Rab12 or GST-Rab12(K71R), immobilized on glutathione agarose beads, in the presence of 0.5 mM GTPγS. Bound proteins were resolved by SDS-PAGE and analysed by immunoblotting with anti-T7 antibodies. Input=10% of total protein. A representative blot is shown. FIG. 2C presents the quantification of the blots by the ImageJ software. Binding is presented as % of total input. Results are the average±SEM derived from three independent experiments. *P(RILP-L1: GST-Rab12/GST-Rab12(1(71R)=0.014, **P(RILP-L2: GST-Rab12/GST-Rab12(K71R)=0.0013.

FIGS. 3A, 3B, 3C, 3D and 3E present in silico modelling of mouse Rab12 and Rab12-RILP dimer complex structures.

FIGS. 3A and 3B show an in silico model of the structure of GDP-bound (pink) and GTP-bound Rab12 (blue). Highlighted are residues that are affected by the conformational changes that occur during Rab12 activation cycle, K-71 (grey), S-72 to K-79 (yellow) and E-101 to R-112 (green). R-50 is shown in orange. Figures were generated using Pymol. FIG. 3C shows the RMSF of Rab12 and FIG. 3D shows the RMSF of the RILP homodimer, during MD simulation. The two predicted Rab12 interfaces are marked in green and purple and the RILP interface in yellow. FIG. 3E shows a model for RILP homodimer interaction with GTP-bound Rab12. RILP monomers are shown in red and light pink. Predicted interfaces in Rab12 are shown in green and purple and the predicted interface in RILP in yellow.

FIGS. 4A, 4B, 4C, 4D and 4E present predicted interactions within the first interface of the mouse Rab12-RILP complex.

FIG. 4A shows that a medium strength salt bridge is generated between Rab12 D-77 and RILP residue R-234, and a stronger interaction between D-77 and K-238 present within RILP RHD (yellow) of same monomer (red). FIG. 4B shows that a stable interaction occurs between F-78 and K-238. FIG. 4C shows that Rab12 V-74 interacts with L-227 of same RILP monomer. FIG. 4D shows that Rab12 K-71 is pulled away from RILP residues E-226 and Q-229. FIG. 4E shows that K-71 forms an intramolecular hydrogen bond with D-96.

FIGS. 5A, 5B, 5C, 5D and 5E show predicted interactions within the second interface of the mouse Rab12-RILP complex.

FIG. 5A shows the positional interactions between F-103 and 1-106 of the second Rab12 interface (purple) with RILP residue L-231 that resides in RILP RHD (yellow). The relative position of S-105 is also depicted. FIG. 5B shows that F-103 also interacts with L-227 of same RILP monomer (red). FIG. 5C shows that a hydrogen bond is formed between Rab12 Y-110 and residue E-236 at the RHD of same RILP monomer. FIG. 5D shows that R-112 interacts with residue T-287 of the second RILP monomer (light pink). FIG. 5E shows that E233 located in one RILP monomer interacts with residue R234 of the second monomer.

FIGS. 6A and 6B show the dynamics of Rab12-RILP interactions.

FIG. 6A shows snapshots illustrating the dynamics of interactions within the first interface of the Rab12-RILP complex. Rab12 amino acids that form the first interface (green) are coloured in purple, and RILP RHD (yellow) amino acids that bind FIG. 5B shows snapshots illustrating the dynamics of interactions within the second interface of the Rab12-RILP complex. Rab12 amino acids that form the second interface (purple) are coloured in orange, and RILP RHD (yellow) amino acids that bind Rab12 are coloured in dark grey. RILP monomers are coloured in red and light pink.

FIGS. 7A and 7B show the mutational analysis that supports RILP RHD involvement in mediating Rab12 binding.

FIG. 7A shows the results of a pulldown experiment, in which RBL cell lysates (500 μg) derived from RBL cells transfected with 35 μg of plasmids encoding either T7-tagged RILP, or T7-tagged RILP(L231A), or T7-tagged RILP(E233A), or T7-tagged RILP(N235A) RHD mutants, were incubated for 18 h at 4° C., in the presence of 0.5 mM GTPγS with 20 of immobilized GST or GST-Rab12. Bound proteins were eluted by sample buffer, and analyzed by SDS-PAGE and immunoblotting, using monoclonal antibodies directed against T7. Input=10% of total protein. A representative blot is shown. FIG. 7B shows the quantification of the amount of pulled down proteins using the ImageJ software. The results are the average pulldown±SEM derived from three independent experiments. *P[(T7-RILP/T7-RILP(L231A)]=0.0480, *P[T7-RILP/T7-RILP(E233A)]=0.0498.

FIGS. 8A and 8B show that RILP RHD mutants differently affect the SG distribution in MCs.

FIG. 8A shows the cellular distribution of the SGs in RBL cells that were transiently co-transfected with 15 μg of plasmid encoding NPY-mRFP, 15 μg of pEGFP-C1-Rab12 and 20 μg of either empty vector or pEF-T7-RILP, pEF-T7-RILP(N235A), pEF-T7-RILP(L231A) or pEF-T7-RILP(E233A), as indicated. After 24 h the cells were fixed and immunostained with monoclonal antibodies directed against T7, followed by Hilyte Plus 647-conjugated goat anti-mouse IgG. Cells were visualized by confocal microscopy. Bar=10 μm. FIG. 8B shows the quantitative analyses of the incidence of cells that display perinuclear SGs were based on the imaging of 20-35 cells, derived from three separate experiments. A single factor ANOVA was performed followed by a Bonferroni corrected post-hoc T-test, ***P[T7-RILP/control]=3E-9, ***P[T7RILP(L231A)/T7-RILP]=2E-6, ***P[T7RILP(N235A)/control]=1,4E-8, ***P[T7RILP(E233A)/control]=3.5E-9

FIGS. 9A and 9B show that Rab12 recruits RILP-L1 and RILP-L2 to its perinuclear location.

FIG. 9A shows the cellular location of RILP-L1 in RBL cells that were transiently co-transfected with 15 μg of plasmid encoding NPY-mRFP, 20 μg of pEF-T7-RILPL-1 and 15 μg of either pEGFP-C1 or pEGFP-C1-Rab12, as indicated. After 24 h, cells were fixed and immunostained with monoclonal antibodies directed against T7, followed by Hilyte Plus 647-conjugated goat anti-mouse IgG. FIG. 9B shows the cellular location of RILP-L2 in RBL cells that were transiently co-transfected with 15 μg of plasmid encoding NPY-mRFP, 20 μg of pEF-T7-RILPL-2 and 15 μg of either pEGFP-C1 or pEGFP-C1-Rab12, as indicated. Cells were processed as described above. Cells were visualized by confocal microscopy. Bar=10 μm.

FIGS. 10A and 10B show Rab12 phosphorylation in RBL cells.

FIG. 10A shows the phosphorylation level of Rab12 in untreated (UT) RBL cells, or in cells that were activated with antigen (IgE/Ag), or with a combination of calcium ionophore (Ion) and the phorbol ester (TPA). For this purpose, RBL cells were seeded in 10 cm plates overnight in growth medium or medium containing DNP-directed IgE. Next day, cells were washed three times with Tyrode's buffer (20 mM Hepes pH 7.4, 137 mM NaCl, 2.7 mM KCl, 1.8 mM CaCl₂), 1 mM MgCl₂, 0.4 mM NaH₂PO₄, 5.6 mM glucose, and 0.1% BSA). Then cells were either left untreated (UT), or treated with 50 ng/ml of the antigen DNP-HSA (IgE/Ag), or with a combination of 1 μM 4-bromo-calcium ionophore A23187 (Ion) and 50 nM of the phorbol ester (TPA), for 30 minutes at 37° C. Cells were subsequently washed three times with PBS and lysed with lysis buffer containing phosphatase inhibitors. Samples were centrifuged and proceeded for western blotting with anti phosphoRab12 antibodies and reprobed with anti Rab12 antibodies. A representative blot is shown. FIG. 10B shows the quantification of the amount of phosphorylated and total Rab12 using the ImageJ software. The results are the ratio of phosphoRab12 to total Rab12. Similar results were obtained in three separate experiments.

FIGS. 11A and 11B show the effect of inhibitors on Rab12 phosphorylation in bone marrow-derived MCs (BMMCs).

FIG. 11A shows the phosphorylation state of Rab12 in BMMCs that were activated by a combination of a calcium ionophore (Ion) and the phorbol ester (TPA) in the absence or presence of the indicated inhibitors. BMMCs were seeded in 10 cm plates overnight in growth medium or medium containing 400 nM TPA. Next day cells were collected and washed three times with Tyrode's buffer in Eppendorf tubes. Cells were subsequently incubated for 30 minutes at 37° C. with vehicle (0.1% DMSO) or with 10 μM GSK2578215A (GSK), 1 μM Go6976, 2 mM EGTA or 1 μM MRT68921, as indicated. Cells were then left untreated (UT) or treated for additional 30 minutes at 37° C., with a combination of 1 μM 4-bromo-calcium ionophore A23187 (Ion) and 50 nM of the phorbol ester (TPA), in the absence or presence of the inhibitors. Cells were then washed three times with PBS, lysed with lysis buffer containing phosphatase inhibitors and cell lysates analysed by western blotting with anti phosphoRab12 antibodies followed by reprobing with anti Rab12 antibodies. A representative blot is shown.

FIG. 11B shows the quantification of the amount of phosphorylated and total Rab12 using the ImageJ software. The results are the relative ratio of phosphorylated to total Rab12. Similar results were obtained in two separate experiments.

FIG. 12 shows Rab12 phosphorylation in SH-SY5Y cells

FIG. 12 shows the phosphorylation of Rab12 in SH-SY5Y cells that were activated by a combination of a calcium ionophore (Ion) and the phorbol ester (TPA). Cells were seeded overnight in 10 cm culture plates. After three washes in Tyrode's buffer, cells were either left untreated (UT) or stimulated in the same buffer for 30 minutes at 37° C. with a combination of 1 μM 4-bromo-calcium ionophore A23187 (Ion) and 50 nM of the phorbol ester (TPA). Cells were lysed and processed for western blotting with anti phosphoRab12 antibodies, followed by reprobing with anti Rab12 antibodies. A representative blot is shown. Similar results were obtained in two separate experiments.

FIGS. 13A and 13B shows Rab12 phosphorylation in rotenone-treated PC12 cells:

FIG. 13A shows an immunoblot of PC12 cell lysates derived from cells that were either left untreated or incubated for 48 hours at 37° C. with 1 μM LY333531 or 10 μM GSK2578215A in the presence or absence of 100 nM rotenone. Cells that were incubated in the absence of rotenone were then left untreated or incubated with a combination of 1 μM 4-bromo-calcium ionophore A23187 (Ion) and 50 nM of the phorbol ester (TPA) for 30 minutes at 37° C. Cells were then washed three times with PBS, lysed with lysis buffer containing phosphatase inhibitors and cell lysates analysed by western blotting with anti phosphoRab12 antibodies followed by re-probing with anti GAPDH antibodies. FIG. 13B shows the quantification of the blot using the ImageJ software. The results are the fold increase in Rab12 phosphorylation based on the ratio of phosphorylated Rab12 to GAPDH. Similar results were obtained in two separate experiments.

FIG. 14: shows Rab12 and phosphoRab12 pulldown assays

FIG. 14 shows the results of a pulldown experiment, in which RBL cells were seeded in 10 cm plates overnight in growth medium or medium containing DNP-specific IgE. Next day, cells were washed three times with Tyrode's buffer and either left untreated (UT) or treated with a combination of 1 μM 4-bromo-calcium ionophore A23187 (Ion) and 50 nM of the phorbol ester (TPA), or with 50 ng/ml DNP-HSA (Ag) for 30 minutes at 37° C., as indicated. Cells were then lysed and 500 μg of cell lysate were incubated overnight at 4° C. with either GST, GST-RILP, GST-RILP-L1 or GST-RILP-L2, immobilized on glutathione agarose beads, as indicated. At the end of the incubation period, beads were sedimented by centrifugation at 5000×g for 5 minutes at 4° C., washed four times with buffer containing 50 mM Hepes pH 7.4, 150 mM NaCl, 1 mM MgCl₂, 0.2% Triton X-100, protease inhibitor mixture, 1 mM PMSF, 2 mM Na₃VO₄, 10 mM NaPPi and 80 mM β-glycerophosphate and suspended in 1× sample buffer and boiled for 7 minutes. Proteins were resolved by SDS-PAGE and analyzed by immunoblotting with the anti-phosphoRab12 antibodies, followed by reprobing with anti Rab12 antibodies, as indicated. Representative blots are shown. Similar results were obtained in two separate experiments.

FIG. 15 shows a model for the regulation of Rab12 connectivity by phosphorylation.

According to this model, Rab12 preferably interacts with some effectors, such as RILP, in its non-phosphorylated form, while it preferably interacts with other effectors, such as RILP-L1 and RILP-L2, in its phosphorylated form. Rab12 conversions between its non-phosphorylated and phosphorylated forms are dictated by the kinases LRRK2, protein kinase C (PKC) and Ulk1, which based on literature results (for LRRK2) and our results (PKC and Ulk1) mediate Rab12 phosphorylation. This conversion is also regulated by yet unidentified protein phosphatases.

FIG. 16 shows Rab12 predicted map of interactions

Interaction sites between human Rab12 and human RILP were predicted based on the in silico modelling and Molecular dynamics simulations of the mouse Rab12-RILP complex, described in FIGS. 2-9 and in Table 1. Interaction sites between phosphoRab12 and RILP-L2 were predicted based on the crystal structure of the complex of phosphoRab8 and RILP-L2.

FIGS. 17A and 17B show peptide inhibition of Rab12 interaction with RILP

FIG. 17A shows the results of a pulldown experiment, in which 5 μg of control GST and GST-RILP, immobilized on glutathione agarose beads, were incubated for 4 hours at 4° C. with 100 μM of either peptide Rab121 or peptide Rab125 or their combination, followed by overnight incubation with 500 μg of RBL cell lysates. At the end of the incubation period, beads were sedimented by centrifugation at 5000×g for 5 min at 4° C., washed four times with 50 mM Hepes pH 7.4, 150 mM NaCl, 1 mM MgCl₂, 0.2% Triton X-100, protease inhibitor mixture, 1 mM PMSF, 2 mM Na₃VO₄, 10 mM NaPPi and 80 mM β-glycerophosphate and suspended in 1× sample buffer and boiled for 7 minutes. Proteins were resolved by SDS-PAGE and analyzed by immunoblotting with anti Rab12 antibodies. FIG. 17B shows the relative amount of pulled down Rab12 based on quantification using the ImageJ software. The results are the average pulldown±SEM derived from two independent experiments.

FIG. 18 shows the impact of the TAT-125 peptide on SG distribution as well as on the morphological changes imposed by rotenone treatment.

FIG. 18 shows the effect of TAT-conjugated peptide 125 on the cellular distribution of the SGs, in PC12 cells that express a constitutively active mutant of Rab12, and on the cell morphology and primary cilia size of rotenone treated cells. Cells (4×10⁴ cells/well) were seeded onto 12 mm round glass coverslips in a 24-well plate. Next day, cells were transiently co-transfected using lipofectamine 2000 with 500 ng of plasmid encoding NPY-mRFP and 1000 ng of pEGFP-C1-Rab12(Q100L), a GTP-locked, constitutively active mutant of Rab12 (herein: CA Rab12). After transfection, cells were either left untreated (UT) or incubated for 48 hours at 37° C. with 100 nM rotenone. After 48 hours, cells were incubated for an additional hour at 37° C. with or without 100 μM of TAT-125 peptide (i.e. YGRKKRRQRRRGGEACKSTVGVDFKIKT, SEQ ID NO: 14), as indicated. Cells were then fixed and immunostained with polyclonal antibodies directed against Arl13b (primary cilium marker), followed by Hilyte Plus 647-conjugated goat anti-rabbit IgG. Cells were visualized by confocal microscopy. The right panels are the overlap of the confocal images on the corresponding brightfield.

FIGS. 19A and 19B show that phosphorylated Rab12 is predicted to have higher affinity to RILP-L2 than to RILP.

FIG. 19A shows that S106 in human Rab12 (S195 in mouse Rab12) is capped by the arginines. Given the high pka and thus positive charge of the R's residue, they are predicted to stabilize the negatively charged phospho serine and contribute to the PPI of Rab12-RILP-L2. FIG. 19B shows that the arginine residue in RILP-L2 is replaced by Glutamic acid, E249, in RILP interface which imparts repulsive interaction when S106 in Rab12 is phosphorylated. *Residue numbers are according to the relevant PDBs structures (human). Homology modeling was generated based on PDB structures 6SQ2 for Rab12/RILP-L2 and 1YHN for Rab7/RILP.

### SUMMARY OF THE INVENTION

In some embodiments, there is provided a polypeptide comprising five (5) or more amino acids of an amino acid sequence having at least 70% identity to the amino acid sequence as set forth in amino acid sequence ID No. 1; and five (5) or more amino acids of an amino acid sequence having at least 70% identity to amino acid sequence ID No. 2; wherein amino acid sequence ID No. 1 and amino acid sequence ID No. 2 refer to Interface II and Interface I, respectively, and are derived from human Rab12 protein.

In some embodiments, the polypeptide further comprises a linker between the five (5) or more amino acids of an amino acid sequence having at least 70% identity to the amino acid sequence as set forth in amino acid sequence ID No. 1; and five (5) or more amino acids of an amino acid sequence having at least 70% identity to amino acid sequence ID NO: 2.

In some embodiments, amino acid sequence ID No. 1 is ERFNSITSAYYR (SEQ ID. NO: 1) and amino acid sequence ID No. 2 is amino acid CKSTVGVDFKI (SEQ ID NO: 2).

In some embodiments, amino acid sequence ID No. 1 comprises the amino acids at position 71-81 of human Rab12 and wherein amino acid sequence ID No. 2 comprises the amino acids at position 102-113 of human Rab12

In some embodiments, the polypeptide comprising 5, 6, 7, 8, 9, 10, 11 or 12 amino acids that are derived from the amino acid sequence ID NO: 1 and 5, 6, 7, 8, 9, 10 or 11 amino acids that are derived from the amino acid sequence ID NO:2.

In some embodiments, one or more of the serine (S) of the polypeptide is replaced by another amino acid.

In some embodiments, another amino acid is aspartate, glutamate, alanine or Serine-phosphate.

In some embodiments, the peptide having at least 70% identity derived from Interface II is ERFNSITSAYYRSAK (peptide Rab121) (SEQ ID NO: 4), ERFNDITSAYYRSAK (peptide Rab122) (SEQ ID NO: 5), ERFNSITSAYYRDAK (peptide Rab123) (SEQ ID NO: 6) or ERFNDITSAYYRDAK (peptide Rab124) (SEQ ID NO: 7).

In some embodiments, the peptide having at least 70% identity derived from Interface I is EACKSTVGVDFKIKT (peptide Rab125) (SEQ ID NO: 8).

In some embodiments, the linker has between 1-20 amino acids.

In some embodiments, there is provided a composition comprising the polypeptide of the invention and a pharmaceutically acceptable carrier.

In some embodiments, the polypeptide or the composition comprising the same may be used in treating a disease associated with imbalance of Rab12 phosphorylation or caused by imbalance of Rab12 interactions with its effectors via Interface I or Interface II or both in a subject in need thereof.

In some embodiments, there is provided a nucleic acid molecule encoding the polypeptide of the invention.

In some embodiments, there is provided a vector comprising the nucleic acid encoding the polypeptide of the invention and one or more regulatory sequences.

In some embodiments, the nucleic acid or the vector of the invention are used in treating a disease associated with imbalance of Rab12 phosphorylation or caused by imbalance of Rab12 interactions with its effectors via Interface I or Interface II or both in a subject in need thereof.

In some embodiments, there is provided a method of treating a subject suffering from a disease caused by imbalance of Rab12 interactions with its effectors via Interface I or Interface II or both comprising the steps of administering to the subject an agent that affect the affinity of Rab12 to its effectors via Interface I or Interface II.

In some embodiments, the agent is a polypeptide comprising five (5) or more amino acids of an amino acid sequence having at least 70% identity to the amino acid sequence as set forth in amino acid sequence ID NO: 1; five (5) or more amino acids of an amino acid sequence having at least 70% identity to amino acid sequence ID NO: 2; or a combination thereof, wherein amino acid sequence ID NO:1 and amino acid sequence ID NO: 2 are derived from human Rab12 protein. In some embodiments, if the polypeptide is a polypeptide comprising five (5) or more amino acids of an amino acid sequence having at least 70% identity to the amino acid sequence as set forth in amino acid sequence ID NO: 1; and five (5) or more amino acids of an amino acid sequence having at least 70% identity to amino acid sequence ID NO: 2, the polypeptide further comprises a linker.

In some embodiments, there is provided method of treating a subject suffering from a disease caused by imbalance of Rab12 phosphorylation comprising the steps of administering to the subject an agent that affect the affinity of Rab12 to its effectors via Interface I or Interface

II.

In some embodiments, the effectors are RILP, RILP-like 1 (RILP-L1) and RILP-Like 2(RILP-L2).

In some embodiments, the disease caused by imbalance of Rab12 phosphorylation or caused by imbalance of Rab12 interactions with its effectors via Interface I or Interface II or both is one or more of amyotrophic lateral sclerosis (ALS), Parkinson's disease (PD), glaucoma, inflammatory disease, Crohn's disease, neurodegenerative disease, musician's dystonia (MD) and writer's dystonia (WD), leprosy, Autism spectrum disorder or tuberculosis.

In some embodiments, the agent is a polypeptide comprising five (5) or more amino acids of an amino acid sequence having at least 70, 75, 80, 85, 90, 95, 97 99% identity to the amino acid sequence as set forth in amino acid sequence ID NO: 1; five (5) or more amino acids of an amino acid sequence having at least 70, 75, 80, 85, 90, 95, 97 99% identity to amino acid sequence ID NO: 2; or a combination thereof, wherein amino acid sequence ID NO: 1 and amino acid sequence ID NO: 2 are derived from human Rab12 protein. In some embodiments, if the polypeptide is a polypeptide comprising five (5) or more amino acids of an amino acid sequence having at least 70, 75, 80, 85, 90, 95, 97 99% identity to the amino acid sequence as set forth in amino acid sequence ID NO: 1; and five (5) or more amino acids of an amino acid sequence having at least 70, 75, 80, 85, 90, 95, 97 99% identity to amino acid sequence ID NO: 2, the polypeptide further comprises a linker.

In some embodiments, there is provided a polypeptide comprising 5, 6, 7, 8, 9, 10 or 11 amino acids of an amino acid sequence having at least 70, 75, 80, 90, 91, 92, 93, 94, 95, 96, 97, 98 or 99% identity or identical to the amino acid sequence as set forth in amino acid sequence RPRPTLQELRD (SEQ ID NO: 3).

In some embodiments, the polypeptide comprises the sequence set forth in

(SEQ ID NO: 9)
KPRHPENHLRK;
(SEQ ID NO: 10)
KPRHWEQTLRN;
(SEQ ID NO: 11)
KPRHWEQLLR;
(SEQ ID NO: 12)
LPRNMRQSLRI;
(SEQ ID NO: 13)
KPRHWEQTLRK;
(SEQ ID NO: 17)
KPRHKLQHLRK;
(SEQ ID NO: 18)
KPRHPEQHLRK;
(SEQ ID NO: 19)
KPRHPLQHLRK;
(SEQ ID NO: 20)
KPRHPEQTLRK;
(SEQ ID NO: 21)
KPRKDSQSLRF;
(SEQ ID NO: 22)
KPRHWEQLLRN;
(SEQ ID NO: 23)
KPRHKSTSLRD;
(SEQ ID NO: 24)
KPRKDLQSLRF;
(SEQ ID NO: 25)
LPRNARQNLRI;
(SEQ ID NO: 26)
HPRNHRQALRI;
(SEQ ID NO: 27)
HPRNMRQALRI;
(SEQ ID NO: 28)
LPRNARQSLRI;
(SEQ ID NO: 29)
HPRNMRQSLRI;
(SEQ ID NO: 30)
IPRNLRHNLRD;
(SEQ ID NO: 31)
LPRNARHELRS;
(SEQ ID NO: 32)
LPRNLRQNLRD;
and
(SEQ ID NO: 33)
VPRNLRHNLRD.

In some embodiments, the peptide for use in treating a disease associated with imbalance of Rab12 phosphorylation or caused by imbalance of Rab12 interactions with its effectors via Interface I or Interface II or both in a subject in need thereof.

In some embodiments, there is provided a nucleic acid molecule encoding the polypeptide polypeptide comprising 5, 6, 7, 8, 9, 10, or 11 of an amino acid sequence having at least 70, 75, 80, 90, 91, 92, 93, 94, 95, 96, 97, 98 or 99% identity to the amino acid sequence as set forth in amino acid sequence RPRPTLQELRD (SEQ ID NO: 3) or of any one of the polypeptide set forth in sequences SEQ ID NOs: 9, 10, 11, 12, 13, 17, 18, 19, 20, 21, 22, 23, 24, 25, 26, 27, 28, 29, 30, 31 32 or 33. In some embodiments, there is provided a vector comprising the nucleic acid and one or more regulatory sequences.

In some embodiments, there is provided a method for treating one or more of amyotrophic lateral sclerosis (ALS), Parkinson's disease, glaucoma, inflammatory disease, Crohn's disease, neurodegenerative disease, dystonia, musician's dystonia (MD) and writer's dystonia (WD), leprosy, Autism spectrum disorder or tuberculosis comprising the step of administering to a subject in need a therapeutically effective amount of the polypeptide comprising 5, 6, 7, 8, 9, 10 or 11 amino acids of an amino acid sequence having at least 70, 75, 80, 90, 91, 92, 93, 94, 95, 96, 97, 98 or 99% identity to the amino acid sequence as set forth in amino acid sequence RPRPTLQELRD (SEQ ID NO: 3) or of any one of the polypeptide set forth in sequences SEQ ID NOs: 9, 10, 11, 12, 13, 17, 18, 19, 20, 21, 22, 23, 24, 25, 26, 27, 28, 29, 30, 31 32 or 33 or or the nucleic acid or the vector encoding the same.

In some embodiments, the peptide or the chimeric peptide of the invention is linked to an internalization peptide or is lapidated or is encapsulated thereby facilitating passage of the peptide across a cell membrane or the blood brain barrier.

### DETAILED DESCRIPTION OF THE EMBODIMENTS OF THE INVENTION

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. The polynucleotides may be, for example, or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions. As used herein, nucleotides (A, G, C or T) and nucleotide sequences are marked in lowercase letters (a, g, c or t).

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the polypeptide, can contain modification, such as but be not limited only to, glycosylation, phosphorylation or disulfide bond shape. Also provided are conservative amino acid variants of the peptides and protein molecules disclosed herein. Variants according to the invention also may be made that conserve the overall molecular structure of the encoded proteins or peptides. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. As used herein, Amino acids and peptide sequences are marked using conventional Amino Acid nomenclature (single letter or 3-letters code). For example, amino acid “Serine” may be marked as “Ser” or “S” and amino acid “Cysteine” may be marked as “Cys” or “C”.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A or a) forms a base pair with thymine (T or t) and guanine (G or g) with cytosine (C or c). In RNA, thymine is replaced by uracil (U or u). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors, plasmids but should not be seen as being limited thereto. The term “regulatory sequence” in some embodiments, refers to DNA sequences, which are necessary to effect the expression of coding sequences to which they are operably linked (connected/ligated). The nature of the regulatory sequences differs depending on the host cells. For example, in prokaryotes, regulatory/control sequences may include promoter, ribosomal binding site, and/or terminators. For example, in eukaryotes regulatory/control sequences may include promoters, terminators enhancers, transactivators and/or transcription factors. A regulatory sequence which is “operably linked” to a coding sequence is ligated in such a way that expression of the coding sequence is achieved under suitable conditions. In some embodiments, a “Construct” or a “DNA construct” refer to an artificially assembled or isolated nucleic acid molecule which comprises a coding region of interest and optionally additional regulatory or non-coding sequences.

As used herein, the term “vector” refers to any recombinant polynucleotide construct (such as a DNA construct) that may be used for the purpose of transformation, i.e. the introduction of heterologous DNA into a host cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced. The term “Expression vector” refers to vectors that have the ability to incorporate and express heterologous nucleic acid fragments (such as DNA) in a foreign cell. In other words, an expression vector comprises nucleic acid sequences/fragments (such as DNA, mRNA), capable of being transcribed or expressed in a target cell. Many viral, prokaryotic and eukaryotic expression vectors are known and/or commercially available. Selection of appropriate expression vectors is within the knowledge of those having skill in the art. The expression vectors can include one or more regulatory sequences.

As used herein, a “primer” defines an oligonucleotide which is capable of annealing to (hybridizing with) a target nucleotide sequence, thereby creating a double stranded region which can serve as an initiation point for DNA synthesis under suitable conditions.

As used herein, the term “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, injection, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

The terms “upstream” and “downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

As used herein, the term “treating” includes, but is not limited to one or more of the following: abrogating, ameliorating, inhibiting, attenuating, blocking, suppressing, reducing, delaying, halting, alleviating or preventing symptoms associated with a condition. Each possibility represents a separate embodiment of the invention. In some embodiments, the condition or the disease are associated with changes in the connectivity of Rab12 with its effectors. In some exemplary embodiments, the condition may be selected from amyotrophic lateral sclerosis (ALS), Parkinson's disease (PD), glaucoma, inflammatory disease, Crohn's disease, neurodegenerative disease, musician's dystonia (MD) and writer's dystonia (WD), leprosy or tuberculosis.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

By “pharmaceutically acceptable” is meant a material that is not biologically or otherwise undesirable, i.e., the material can be administered to an individual along with the selected compound without causing any undesirable biological effects or interacting in a deleterious manner with any of the other components of the pharmaceutical composition in which it is contained.

In this specification and in the claims, which follow, reference will be made to a number of terms which shall be defined to have the following meanings: “Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

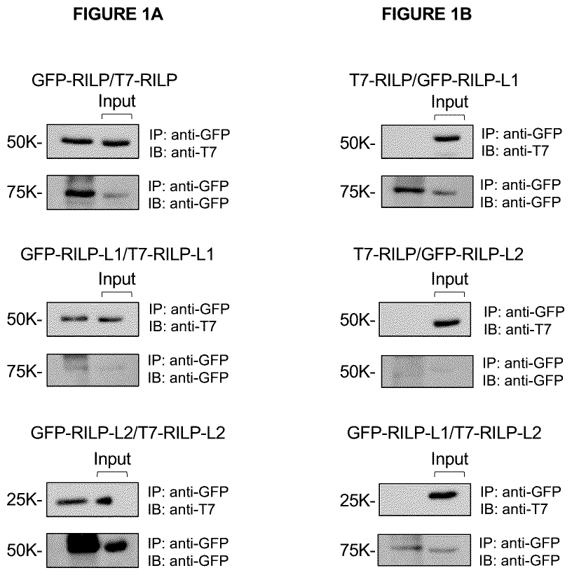

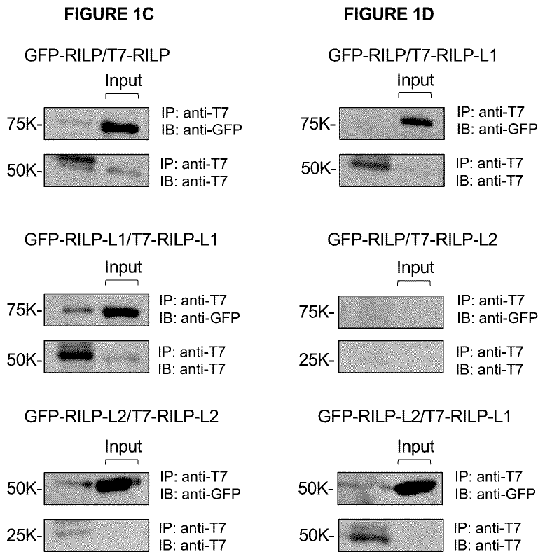

Given the role of Rab effectors in mediating Rab functions, biochemical and in silico tools to decipher Rab12 interactions with its RILP family effectors were used. FIG. 1 shows that Rab12 independently interacts with the three members of the RILP family, RILP, RILP-like 1 (RILP-L1) and RILP-like 2 (RILP-L2). In some embodiments, Rab12 binding site was delineated and lysine-71 in mouse Rab12 was identified as critical for its interactions with RILP-L1 and RILP-L2, but not RILP. Based on structural modelling of the Rab12-RILP complex, a ternary complex consisting of a RILP homodimer and one molecule of GTP-bound Rab12 was suggested, that interacts, via the switch I and switch II regions, with the RILP homology domain (RHD) of one RILP monomer and a C-terminal threonine in the other monomer. Mutational analyses of RILP RHD confirm the involvement of this domain in Rab12 binding and the regulation of mast cells (MC) secretory granules (SG) transport. The results provided structural and functional insights into the Rab12-RILP complex on the basis of which new tools could be generated for decoding Rab12 connectivity. Based on structural modelling of the Rab12-RILP-L2 complex, a ternary complex consisting of a RILP-L2 homodimer and one molecule of GTP-bound Rab12 was suggested, in which phosphoRab12 interacts with the RILP-L2 region encompassing the arginine. residues 130 to 139. Rab12, a member of the Rab family of GTPases, was identified as regulator of the spatiotemporal distribution of the secretory granules (SGs) in triggered mast cells (MCs). The latter are key regulatory cells of the immune system that are best known for their critical role in allergy and anaphylaxis, though their strategic positioning at the interfaces with the external environment, such as in the skin, respiratory and digestive systems, alongside their responsiveness to multiple external triggers, including the allergic, immunoglobulin E (IgE)-dependent and FcεRI-mediated atopic trigger, a variety of neuropeptides, drugs, toxins and cell to cell contact, mark them as sentinel cells in first line host defense. Both the physiological and pathophysiological functions of the MCs, in health and disease, are primarily mediated by their release, by regulated exocytosis, of multiple inflammatory mediators that are pre-formed and stored in the SGs, thus assigning these organelles a central role in executing MC responses. To release their content, the SGs need to move to, and fuse with the plasma membrane, a kinesin-1 driven process, that is regulated by the small GTPase Rab27b. However, MC SGs were shown to move bidirectionally, and it was recently demonstrated that Rab12 stimulates microtubule (MT) dependent, minus end transport of the SGs in triggered cells, by recruiting the RILP-dynein protein complex. Rab12 has been previously implicated in controlling constitutive functions, such as controlling the traffic of the transferrin receptor from the recycling endosomes to lysosomal degradation and inducing autophagy by facilitating the degradation of amino acid transporter PAT4. Indeed, in addition to its interaction with the dynein binding protein RILP, Rab12 also binds the two other members of the RILP family, RILP-Like 1 (RILP-L1) and RILP-Like 2 (RILP-L2). As the latter members of the RILP family lack a dynein binding site, it is reasonable to assume that the functions of their complexes with Rab12 are distinct from the function fulfilled by the Rab12-RILP complex. Therefore, while the precise functions of Rab12 interactions with its RILP-L1 and RILP-L2 effectors are presently unknown, it is anticipated that Rab12 impact on the spatio-temporal distribution of the SGs, and therefore also on exocytosis, would be defined by its relative distribution between its distinct effectors. However, what precisely dictates the balance of Rab12 connectivity is currently unknown. A hint of a possible mechanism emerged from recent studies that identified Rab12 as one of the physiological substrates of the Leucine-Rich Repeat kinase 2 (LRRK2). Moreover, LRRK2-mediated phosphorylation of Rab12 resulted in increased binding of its effectors, RILP-L1 and RILP-L2. LRRK2 is highly expressed in immune cells, in which its function has been linked to inflammation, phagocytosis, macropinocytosis and autophagy. Therefore, the inventors set up to investigate whether phosphorylation of Rab12 plays a role in controlling its distribution between its RILP family effectors, and thereby controlling MC exocytosis. The examples herein demonstrate that Rab2 phosphorylation has opposite effects on its interactions with RILP versus RILP-L1/RILP-L2. Further, Rab12 phosphorylation by protein kinase C by a mechanism that involves the Ulk1/2 kinases, has a similar impact on its connectivity.

All three members of the RILP family of proteins have been shown to interact with the small GTPase Rab12, though the precise modes of their interactions, role and regulation are still by largely poorly resolved. Towards the understanding of the underlying mechanisms of Rab12 interactions, here it mainly focused on Rab12 interactions with RILP, which was recently shown to mediate Rab12-driven, and microtubule dependent, minus-end transport of the SGs in MCs. Three important findings emerged from the study. First, it was shown that RILP interacts with Rab12 independently of RILP-L1 or RILP-L2. It was shown that although all RILP family members can homodimerize, neither of the RILP family proteins can form heterodimers. This finding excludes the possibility of RILP interaction with Rab12 via a complex with RILP-L1, which is the only RILP family member that was recognized as Rab12 partner during a yeast two hybrid screen. The reason for this discrepancy may reflect distinct dependencies of Rab12 interactions on posttranslational modifications. Rab12 was recently identified as a physiological substrate of LRRK2 and its phosphorylation by this kinase stimulated its interaction with RILP-L2, but not its interaction with RILP-L1. Thus, it was envisioned that posttranslational modifications of Rab12 may also regulate its interaction with RILP. This notion gains support from the simulated model of the Rab12-RILP complex, which positions residue S105 in mouse Rab12, which is equivalent to S106 in human Rab12, the phosphorylation site of Rab12 by LRRK2, at one of Rab12-RILP interfaces.

The binding site of Rab12 for RILP-L1 and RILP-L2 was delineated, and identified the lysine residue at position 71 of mouse Rab12, the equivalent of lysine 72 in human Rab12, as critical for these interactions. To the inventors' surprise, though this lysine was chosen as candidate on the basis of its analogous position to the lysine residues implicated in RILP binding by Rab7 and Rab34, replacing this lysine by arginine, which has completely abrogated the pulldown of either RILP-L1 or RILP-L2 by Rab12, has failed to affect Rab12 capacity to pull down RILP. This observation prompted the understanding the structural events that occur during Rab12 interaction with RILP, for which in silico modelling was employed that allowed to gain structural insights at atomic resolution. Intriguingly, although the model was built on the basis of the resolved x-ray structure of the Rab7-RILP dimer complex, molecular dynamics simulations, based on an energy minimized complex structure, revealed some similarities between the Rab12 and Rab7-RILP complexes, but have also outlined significant differences. Unlike the symmetric Rab7-RILP complex, which consists of a central RILP homodimer and two molecules of GTP-bound Rab7, each binding to a single RILP monomer, the model predicts a ternary protein complex between a homodimer of RILP and a single molecule of GTP-bound Rab12 that interacts with both RILP monomers. This ternary complex is held together via multiple bonds that encompass two interfaces in Rab12, that bind to the RHD of one RILP monomer, and a third contact site between an amino acid within the second interface of Rab12 and the second RILP monomer. The first interface of Rab12 interaction with RILP largely replicates the first interface of RILP interactions with Rab7. In both cases, this interface involves the Rab switch I region, comprising amino acids cysteine 70 to leucine 79 in mouse Rab12, the equivalent of acids cysteine 71 to leucine 80 in human Rab12, which is implicated in Rab effector binding, when bound to GTP. Though exceptional in this regard, is the contribution of the lysine residue within the first interface, i.e. K-38 in Rab7, which contributes significantly to Rab7 interaction with RILP, unlike K-71 of Rab12, which is dispensable for Rab12 interaction with RILP, but is rather involved in an intramolecular interaction, mediated by a hydrogen bond with the aspartate residue at position 96, which pulls lysine 71 away from the RILP complex. Also in analogy to the Rab7-RILP complex, the first interface of the Rab12-RILP complex, includes RILP RHD, which was also implicated in mediating RILP interactions with Rab34 and Rab36.

In sharp contrast, though both Rab7 and Rab12 interact with RILP via an additional interface, a number of important differences distinguish between the two. First, the second predicted interface of Rab12, that spans residues phenylalanine 103 to arginine 112, in mouse Rab12 and equivalent to residues phenylalanine 104 to arginine 113, in human Rab12, resides at the conserved RabF3 and RabF4 regions, which similarly to switch I, and consistent with the model, undergo positional changes during the Rab12 activation cycle. This is different from the second interface of Rab7 that resides in its hypervariable regions RabSF1 and RabSF4. Therefore, unlike Rab7, the second interface of Rab12 is also predicted to bind RILP in a GTP-dependent fashion. Second, unlike Rab7, the second interface of Rab12 also involves the RILP RHD, similarly to the first interface. In this respect, Rab12 interaction with RILP replicates RILP RHD interaction with Rab36, that involves the switch II region of Rab36. Finally, the second interface of Rab12 also forms contact with the second monomer of the RILP dimer. Therefore, the structural features of the Rab12-RILP complex are unique and are likely to be subjected to distinct modes of regulation, consistent with the distinct function of this complex, which mediates retrograde transport of the SGs in cells, whose lysosomes are likely to be transported by the Rab7-RILP complex.

In agreement with the assignment of RILP RHD as RILP site of interaction with Rab12, the mutagenesis and phenotypic analyses demonstrated impairment of both Rab12-RILP complex formation and function following alanine substitution of the leucine residue at position 231 of mouse RILP, the equivalent of leucine residue at position 251 of human RILP. Surprisingly, unlike the RILP(L231A) mutant, RILP(E233A), which also fails to bind Rab12, could cluster the SGs. In view of the fact that this mutant binds Rab36, which based on the Rab screen, induces perinuclear clustering of the SGs, the inventors were prompted to suggest that Rab12 and Rab36 may either function redundantly or play complementary roles in controlling MC SG transport. In this context, it is interesting to note that unlike Rab12, that when overexpressed alone, clusters the SGs only in its constitutively active conformation or in triggered cells, overexpressed Rab36 clusters the SGs also in its wild type form and in resting cells. Since MC SGs move bidirectionally also in resting cells, it is tempting to speculate that Rab36 drives retrograde transport of the SGs in resting cells, while Rab12 drives their transport in activated cells, as part of its negative regulation of MC secretion.

Finally, it was shown that Rab12 acquires its perinuclear location, previously identified as the ERC, regardless to its interactions with its effectors. This is illustrated in the fact that Rab12 is perinuclear also in cells that overexpress the RILP RHD mutants, thus excluding its interaction with RILP in its targeting to the ERC. Similarly, both RILP-L1 and RILP-L2 are primarily cytosolic when overexpressed in the absence of Rab12, but translocate to the perinuclear region, colocalizing with Rab12, in its presence. This contrasts the Rab-effector relationship between RILP-L1 and Rab10, where RILP-L1 localizes to pericentriolar membranes and enhances the accumulation of phosphorylated Rab10 at this site. It will be interesting to explore if Rab12 initiates a Rab cascade by recruiting RILP-L1, that in turn recruits phospho-Rab10. In this case, Rab12 may represent a missing link in the crosstalk between the endocytic recycling compartment (ERC), centrosome and primary cilia.

In conclusion, by combining in silico modelling with mutagenesis analysis, structural insights into the mode of interactions of Rab12 with its RILP effector were provided, on the basis of which, new tools could be developed for further understanding of the regulation and function of the interactions between RILP family members and Rab12.

In view of the fact that RILP is the only effector that has a dynein binding site and is therefore able to control minus end transport of organelles, and because Rab12 was shown to preferably bind RILP-L1 and RILP-L2 in its LRRK2-phosphorylated form, the inventors hypothesized that phosphorylation of Rab12 may have opposite effects on Rab12 interactions with its different effectors. Further, they hypothesize that factors that affect the state of Rab12 phosphorylation would perturb the balance of Rab12 distribution between its different effectors, thereby influencing their Rab12 regulated functions. In some embodiments, such alterations would disturb the cell homeostasis leading to disease. Perturbations of Rab12 balanced connectivity may in some embodiments result from genetic variations in Rab12. In some embodiments, mutations in Rab12 that may have a direct impact on its phosphorylation, are the mutations in Rab12 identified in Musician or Worker dystonia patients, or in any other disease linked with mutations in Rab12 that impact Rab12 phosphorylation. In some embodiments, perturbations of Rab12 balanced connectivity may result from changes in the kinases that phosphorylate Rab12, or phosphatases that dephosphorylate Rab12. Examples for the former are Parkinson's disease, where hyperactivation of LRRK2 leads to hyperphosphorylation of Rab12, or any other inflammatory disease linked with alterations in LRRK2 activity. Examples include leprosy, tuberculosis and inflammatory bowel diseases, in which LRRK2 has been implicated, and in particular in Crohn's disease, for which GWAS has identified LRRK2 as a major susceptibility gene. Additional examples may include pathological conditions linked with hyperactivation of protein kinase C, or Ulk1/2, that based on the results provide an alternative mechanism for Rab12 phosphorylation, leading to similar functional consequences (i.e. preferable interaction with RILP-L2). Other examples may include retinal ganglion cell death-associated with glaucoma in which Rab12 interaction with optineurin is disturbed, though it is presently unknown if phosphorylation affects Rab12 interaction with optineurin.

Functions that might be disturbed upon alterations in the balanced phosphorylation of Rab12 include the Rab12-RILP complex controlled microtubule-dependent, minus end transport of the SGs in activated MCs, that is required for the negative regulation of MC degranulation by Rab12. In some embodiments, Rab12-RILP complex may fulfill a similar role also in other secretory cells, including neuronal cells, in particular in controlling minus end transport of lysosome related organelles (LROs), a family of SG to which the MC SGs belong.

The functions of the Rab12-RILP-L1 and Rab12-RILP-L2 complexes are presently unknown. Rab12 was shown to regulate trafficking of the transferrin receptor from the recycling endosomes to lysosomal degradation, which would impact iron uptake and it also controls autophagy by facilitating the degradation of the amino acid transporter PAT4. Rab12 was also shown to control transport of the Shiga toxin into the cell. However, the effectors that mediate these functions of Rab12 are currently unknown. RILP-L1 and RILP-L2 have been implicated in controlling ciliogenesis and centrosomal organization. RILP-L2 was shown to promote neurite outgrowth by interacting with the actin motor MyoVa. Finally, RILP-L1 was also implicated in the protection of cells from apoptosis, via its interaction with GAPDH. Though it is presently unknown whether these functions involve complex formation with Rab12, it is envisioned that in such case, exaggerated complex formation due to hyperphosphorylation of Rab12 or reduced complex formation due to diminished phosphorylation of Rab12 will result in progression of pathology.

Therefore, peptides designed to manipulate Rab12 connectivity by inhibiting exaggerated complex formation due to hyperphosphorylation of Rab12, or stimulate formation of complexes whose formation is reduced due to hyperphosphorylation of Rab12, will rescue the homeostatic imbalance and attenuate progression of pathology.

In some embodiments, there is provided a polypeptide comprising five (5) or more amino acids of an amino acid sequence having at least 70, 75, 80, 90, 91, 92, 93, 94, 95, 96, 97, 98 or 99% identity to the amino acid sequence as set forth in amino acid sequence ID NO: 1 or a fragment thereof; five (5) or more amino acids of an amino acid sequence having at least 70, 75, 80, 90, 91, 92, 93, 94, 95, 96, 97, 98 or 99% identity to amino acid sequence ID NO: 2 or a fragment thereof; or a combination thereof, i.e. a chimeric peptide comprising a polypeptide comprising five (5) or more amino acids of an amino acid sequence having at least 70, 75, 80, 90, 91, 92, 93, 94, 95, 96, 97, 98 or 99% identity to the amino acid sequence as set forth in amino acid sequence ID NO: 1 or a fragment thereof; and five (5) or more amino acids of an amino acid sequence having at least 70, 75, 80, 90, 91, 92, 93, 94, 95, 96, 97, 98 or 99% identity to amino acid sequence ID NO: 2 or a fragment thereof, wherein amino acid sequence ID NO: 1 and amino acid sequence ID NO: 2 refer to Interface II and Interface I, respectively, and are derived from human Rab12 protein.

In some embodiments, there is provided a polypeptide consisting of five (5) or more amino acids of an amino acid sequence having at least 70, 75, 80, 90, 91, 92, 93, 94, 95, 96, 97, 98 or 99% identity to the amino acid sequence as set forth in amino acid sequence ID NO: 1; five (5) or more amino acids of an amino acid sequence having at least 70, 75, 80, 90, 91, 92, 93, 94, 95, 96, 97, 98 or 99% identity to amino acid sequence ID NO: 2; or a combination thereof, i.e. a chimeric peptide, wherein amino acid sequence ID NO: 1 and amino acid sequence ID NO: 2 refer to Interface II and Interface I, respectively, and are derived from human Rab12 protein.

In some embodiments of the invention, the term “interface II” refers to the amino acid sequence at positions 102-113 of human Rab12, i.e. amino acid sequence ID NO: 1, consisting of ERFNSITSAYYR.

In some embodiments of the invention, the term “interface I” refers to the amino acid sequence at positions 71-81 of human Rab12, i.e. amino acid sequence ID NO: 2 consisting of CKSTVGVDFKI.

In some embodiments, there is provided a chimeric peptide comprising five (5) or more amino acids of an amino acid sequence having at least 70, 75, 80, 90, 91, 92, 93, 94, 95, 96, 97, 98 or 99% identity to the amino acid sequence as set forth in amino acid sequence ID NO: 1; five (5) or more amino acids of an amino acid sequence having at least 70, 75, 80, 90, 91, 92, 93, 94, 95, 96, 97, 98 or 99% identity to amino acid sequence ID NO: 2 and optionally a linker between and wherein amino acid sequence ID NO: 1 and amino acid sequence ID NO: 2 refer to Interface II and Interface I, respectively, and are derived from human Rab12 protein.

In some embodiments, the polypeptide comprises 5, 6, 7, 8, 9, 10, 11 or 12 amino acids that are derived from the amino acid sequence ID NO: 1 and/or 5, 6, 7, 8, 9, 10 or 11 amino acids that are derived from the amino acid sequence ID NO: 2.

In some embodiments, there is provided a polypeptide as described above, wherein at least one serine (S) is replaced by another amino acid. In some embodiments, another amino acid is aspartate, glutamate, alanine or Serine-phosphate.

In some embodiments, the polypeptide comprises ERFNSITSAYYRSAK (peptide Rab121) SEQ ID NO: 4, ERFNDITSAYYRSAK (peptide Rab122) SEQ ID NO: 5, ERFNSITSAYYRDAK (peptide Rab123) SEQ ID NO: 6 or ERFNDITSAYYRDAK (peptide Rab124) SEQ ID NO: 7 or any variant thereof, wherein the variant has at least 70, 75, 80, 90, 91, 92, 93, 94, 95, 96, 97, 98 or 99% identity to ERFNSITSAYYRSAK (peptide Rab121), ERFNDITSAYYRSAK (peptide Rab122) SEQ ID NO: 5, ERFNSITSAYYRDAK (peptide Rab123) or ERFNDITSAYYRDAK (peptide Rab124) SEQ ID NO: 7.

In some embodiments, the polypeptide comprises EACKSTVGVDFKIKT (peptide Rab125) SEQ ID NO: 8 or any variant thereof, wherein the variant has at least 70, 75, 80, 90, 91, 92, 93, 94, 95, 96, 97, 98 or 99% identity to EACKSTVGVDFKIKT (peptide Rab125) SEQ ID NO: 8.

In some embodiments, the linker has between 1-20 amino acids. In some embodiments, the linker has between 2-20 amino acids. In some embodiments, the linker has five amino acids. In some embodiments, the linker has between 3-10 amino acids.

In some embodiments, the linker is a non-peptide linker. In some embodiments, the linker comprises a hydrazide bridge.

In some embodiments, there is provided a method of treating a subject suffering from a disease caused by imbalance of Rab12 interactions with its effectors via Interface I or Interface II or both comprising the steps of administering to the subject an agent that affects the affinity of Rab12 to its effectors via Interface I or Interface II.

In some embodiments an agent that affects the affinity of Rab12 to its effectors via Interface I or Interface II is a polypeptide as described above.

In some embodiments, the agent is a polypeptide comprising five (5) or more amino acids of an amino acid sequence having at least 70, 75, 80, 90, 91, 92, 93, 94, 95, 96, 97, 98 or 99% identity to the amino acid sequence as set forth in amino acid sequence ID NO:1; five (5) or more amino acids of an amino acid sequence having at least 70, 75, 80, 90, 91, 92, 93, 94, 95, 96, 97, 98 or 99% identity to amino acid sequence ID NO:2; or a combination thereof, wherein amino acid sequence ID NO:1 and amino acid sequence ID NO: 2 are derived from human Rab12 protein.

In some embodiments, there is provided a method of treating a subject suffering from a disease caused by imbalance of Rab12 phosphorylation comprising the steps of administering to the subject an agent that affect the affinity of Rab12 to its effectors via Interface I or Interface II.

In some embodiments, Rab12 effectors are RILP, RILP-like 1 (RILP-L1) and RILP-Like 2 (RILP-L2).

In some embodiments, the disease caused by imbalance of Rab12 phosphorylation, or caused by imbalance of Rab12 interactions with its effectors via Interface I or Interface II, or both, is one or more of amyotrophic lateral sclerosis (ALS), Parkinson's disease (PD), glaucoma, inflammatory disease, Crohn's disease, neurodegenerative disease, musician's dystonia (MD) and writer's dystonia (WD), leprosy or tuberculosis.

In some embodiments, the agent is a polypeptide comprising five (5) or more amino acids of an amino acid sequence having at least 70, 75, 80, 90, 91, 92, 93, 94, 95, 96, 97, 98 or 99% identity to the amino acid sequence as set forth in amino acid sequence ID NO: 1; five (5) or more amino acids of an amino acid sequence having at least 70, 75, 80, 90, 91, 92, 93, 94, 95, 96, 97, 98 or 99% identity to amino acid sequence ID NO:2; or a combination thereof, i.e. a chimeric peptide, wherein amino acid sequence ID NO: 1 and amino acid sequence ID NO:2 are derived from human Rab12 protein.

In some embodiments, a therapeutic effect could be achieved by introducing molecules that would strengthen the suppressed interaction or reduce the exaggerated interaction between Rab12 and its effectors. If the disease is linked with hyperphosphorylation of Rab12, such as in the case of PD, where LRRK2 is hyperactive, but not only, because the inventors found that also protein kinase C phosphorylates Rab12, then the aim would be to inhibit Rab12 interactions with RILP-L1/RILP-L2, which take place when Rab12 is phosphorylated, or strengthen the affinity of interaction of Rab12 with RILP, which is mediated by non-phosphorylated Rab12. On the other hand, if the disease is caused because Rab12 phosphorylation is reduced, then inhibition of the interaction of Rab12 with RILP or strengthening its interactions with RILP-L1/RILP-L2 is required.

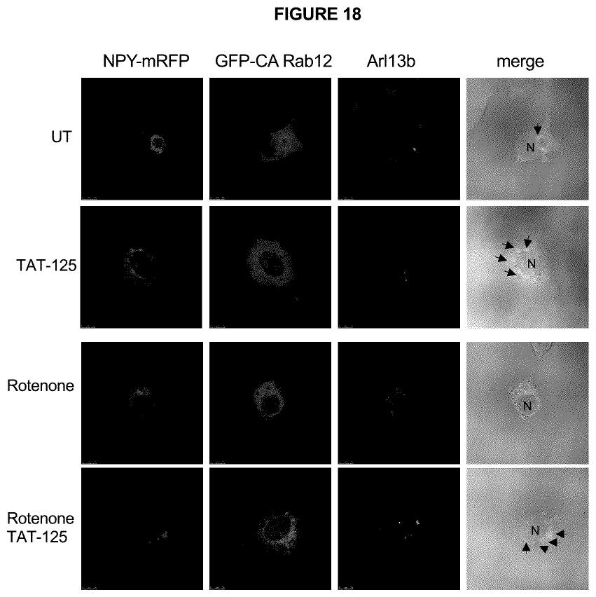

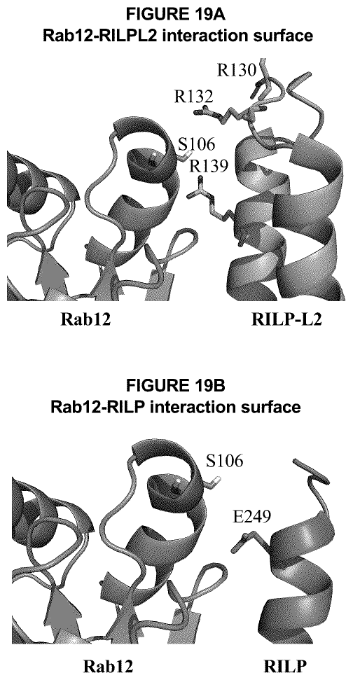

Rab12 plays an important role in functions such as regulation of vesicle transport and autophagy. It has been implicated in diseases such as Parkinson's Disease (PD) and certain types of Dystonias. In its active state Rab12 can interact with three different effectors, two of which, termed RILP-L1 and RILP-L2, play a role in cell sensing of its environment, while the third effector, termed RILP, is involved in transport of organelles. Thus, the balance of Rab12 interactions with its distinct effectors is critical to for normal cell function. As an example, it is shown here (see FIG. 18) that in an in vitro model of PD, that is based on the exposure of PC12 cells to rotenone, an inhibitor of mitochondria complex I, under conditions in which the balance of Rab12 interactions is shifted towards the binding of RILP, by expressing a constitutively active mutant of Rab12, which preferably binds RILP, introduction of a cell permeable version of peptide 125 (i.e. TAT-125), reduces Rab12-RILP interaction, as evidenced by the partial scattering of the clustered SGs, and improves the cell morphology, which was altered by the rotenone treatment. Therefore, these results demonstrate the effectiveness of rational designed peptides in modulating Rab12-effector excessive interactions. Furthermore, Rab12 is physiological substrate of the Leucine-Rich Repeat kinase 2 (LRRK2), and it was shown that indeed non-phosphorylated Rab12 preferably binds RILP, while phosphorylated Rab12 preferably binds RILP-L1/RILP-L2. Therefore, increased activity of LRRK2, as is the case in both familial and idiopathic PD, leading to hyperphosphorylation of Rab12, shifts its interactions towards excessive binding of RILP-L1/RILP-L2. It is suggested that these imbalanced interactions contribute to PD pathogenesis. Thus, restoring the balance of Rab12, by targeting the excessive interactions of its hyperphosphorylated state, will provide a platform for the development of novel therapeutic for arresting PD pathology. This can be achieved through the interfering of phosphoRab12 (pRab12) interaction with RILP-L1/RILP-L2, by unique cell permeable peptides that correspond to the interfaces of phosphoRab12-RILP-L2 complex and can modulate this interaction, thereby acting as therapeutics. Moreover, it was shown that the same serine residue (S106), that is phosphorylated by LRRK2, is also phosphorylated by protein kinase C. Therefore, it is envisioned that diseases linked with hyperactivation of protein kinase C, as is the case of Autism spectrum disorder (ASD) will similarly impair the balance of Rab12 interactions with its RILP family effectors and will therefore also benefit from restoring the balance by peptides that would prevent excessive and pathological complex formation.

In some embodiments, based on the model described in the Examples, peptides predicted to selectively inhibit phosphoRab12 interaction with RILP-L2 while maintaining Rab12 interaction with RILP intact include peptides that share homology with the RILP-L2 derived sequence RPRPTLQELRD (SEQ ID NO: 3), including:

(SEQ ID NO: 9)
KPRHPENHLRK;
(SEQ ID NO: 10)
KPRHWEQTLRN;
(SEQ ID NO: 11)
KPRHWEQLLR;
(SEQ ID NO: 12)
LPRNMRQSLRI;
(SEQ ID NO: 13)
KPRHWEQTLRK;
(SEQ ID NO: 17)
KPRHKLQHLRK;
(SEQ ID NO: 18)
KPRHPEQHLRK;
(SEQ ID NO: 19)
KPRHPLQHLRK;
(SEQ ID NO: 20)
KPRHPEQTLRK;
(SEQ ID NO: 21)
KPRKDSQSLRF;
(SEQ ID NO: 22)
KPRHWEQLLRN;
(SEQ ID NO: 23)
KPRHKSTSLRD;
(SEQ ID NO: 24)
KPRKDLQSLRF;
(SEQ ID NO: 25)
LPRNARQNLRI;
(SEQ ID NO: 26)
HPRNHRQALRI;
(SEQ ID NO: 27)
HPRNMRQALRI;
(SEQ ID NO: 28)
LPRNARQSLRI;
(SEQ ID NO: 29)
HPRNMRQSLRI;
(SEQ ID NO: 30)
IPRNLRHNLRD;
(SEQ ID NO: 31)
LPRNARHELRS;
(SEQ ID NO: 32)
LPRNLRQNLRD;
and
(SEQ ID NO: 33)
VPRNLRHNLRD.

In some embodiments, there is provided a polypeptide comprising 5, 6, 7, 8, 9, 10, 11, 12 or more amino acids of an amino acid sequence having at least 70, 75, 80, 90, 91, 92, 93, 94, 95, 96, 97, 98 or 99% identity to the amino acid sequence as set forth in amino acid sequence RPRPTLQELRD (SEQ ID NO: 3).

In some embodiments, there is provided a method of treating amyotrophic lateral sclerosis (ALS), Parkinson's disease, glaucoma, inflammatory disease, Crohn's disease, dystonia, neurodegenerative disease, musician's dystonia (MD) and writer's dystonia (WD), leprosy, Autism spectrum disorder or tuberculosis comprising the step of administering to a subject in need a therapeutically effective amount of any one of the peptides set forth in sequences SEQ ID NOs: 9, 10, 11, 12, 13, 17, 18, 19, 20, 21, 22, 23, 24, 25, 26, 27, 28, 29, 30, 31, 32 or 33.

In some embodiments, there is provided a method of treating amyotrophic lateral sclerosis (ALS), Parkinson's disease, glaucoma, inflammatory disease, Crohn's disease, dystonia, neurodegenerative disease, musician's dystonia (MD) and writer's dystonia (WD), leprosy, Autism spectrum disorder or tuberculosis comprising the step of administering to a subject in need a therapeutically effective amount of a peptide comprising 5, 6, 7, 8, 9, 10, 11, 12 or more amino acids of an amino acid sequence having at least 70, 75, 80, 90, 91, 92, 93, 94, 95, 96, 97, 98 or 99% identity to the amino acid sequence as set forth in amino acid sequence RPRPTLQELRD (SEQ ID NO: 3).

According to some embodiments, any suitable route of administration to a subject may be used for the nucleic acid, polypeptide or the composition of the invention, including but not limited to, local and systemic routes. Exemplary suitable routes of administration include, but are not limited to: orally, intra-nasally, parenterally, intravenously, topically, enema or by inhalation. According to another embodiment, systemic administration of the composition is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to another embodiment, parenteral administration is transmucosal administration. According to another embodiment, transmucosal administration is transnasal administration. For transmucosal administration, penetrants appropriate to the barrier to be permeated are used in the formulation. Such penetrants are generally known in the art. The preferred mode of administration will depend upon the particular indication being treated and will be apparent to one of skill in the art.

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides.

According to another embodiment, the composition is administered intravenously, and is thus formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

According to another embodiment, administration systemically is through an enteral route. According to another embodiment, administration through an enteral route is buccal administration. According to another embodiment, administration through an enteral route is oral administration. According to some embodiments, the composition is formulated for oral administration.

According to some embodiments, oral administration is in the form of hard or soft gelatin capsules, pills, capsules, tablets, including coated tablets, dragees, elixirs, suspensions, liquids, gels, slurries, syrups or inhalations and controlled release forms thereof.

According to some embodiments, suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

In some embodiments, if desired, disintegrating agents, such as cross-linked polyvinyl pyrrolidone, agar, or alginic acid or a salt thereof, such as sodium alginate, may be added. Capsules and cartridges of, for example, gelatin, for use in a dispenser may be formulated containing a powder mix of the composition of the invention and a suitable powder base, such as lactose or starch.

According to some embodiments, solid dosage forms for oral administration include capsules, tablets, pill, powders, and granules. In such solid dosage forms, the composition of the invention is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

In some embodiments, liquid dosage forms for oral administration may further contain adjuvants, such as wetting agents, emulsifying and suspending agents, and sweetening, flavoring and perfuming agents. According to some embodiments, enteral coating of the composition is further used for oral or buccal administration. The term “enteral coating”, as used herein, refers to a coating which controls the location of composition absorption within the digestive system. Non-limiting examples for materials used for enteral coating are fatty acids, waxes, plant fibers or plastics.

According to some embodiments, administering is administering topically. According to some embodiments, the composition is formulated for topical administration. The term “topical administration”, as used herein, refers to administration to body surfaces. Non-limiting examples of formulations for topical use include cream, ointment, lotion, gel, foam, suspension, aqueous or cosolvent solutions, salve and sprayable liquid form. Other suitable topical product forms for the compositions of the present invention include, for example, emulsion, mousse, lotion, solution and serum.

According to some embodiments, the administration may include any suitable administration regime, depending, inter alia, on the medical condition, patient characteristics, administration route, and the like. In some embodiments, administration may include administration twice daily, every day, every other day, every third day, every fourth day, every fifth day, once a week, once every second week, once every third week, once every month, and the like.

Internalization Elements and Tissue Penetration Elements

The disclosed compositions, peptides, polypeptides, proteins, amino acid sequences, etc. can comprise one or more internalization elements, tissue penetration elements, or both.

Internalization elements and tissue penetration elements can be incorporated into or fused with other peptide components of the composition, such as peptide homing molecules and peptide cargo molecules. Internalization elements are molecules, often peptides or amino acid sequences, that allow the internalization element and components with which it is associated, to pass through biological membranes. Tissue penetration elements are molecules, often peptides or amino acid sequences, that allow the tissue penetration element and components with which it is associated to passage into and through tissue. “Internalization” refers to passage through a plasma membrane or other biological barrier. “Penetration” refers to passage into and through a membrane, cell, tissue, or other biological barrier. Penetration generally involves and includes internalization. Some molecules, such may function as both internalization elements and tissue penetration elements.

Internalization elements include, for example, cell-penetrating peptides. Peptides that are internalized into cells are commonly referred to as cell-penetrating peptides. There are two main classes of such peptides: hydrophobic and cationic. The cationic peptides, which are commonly used to introduce nucleic acids, proteins into cells, include the prototypic cell-penetrating peptides, Tat, and penetratin.

Liposomes

The term “Liposome” as is used herein refers to a structure comprising an outer lipid bi- or multi-layer membrane surrounding an internal aqueous space. Liposomes can be used to package any biologically active agent for delivery to cells. In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film.

The compositions disclosed herein can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art. Pharmaceutical compositions can include carriers, thickeners, diluents, buffers, preservatives, surface active agents and the like in addition to the molecule of choice.

### EXAMPLES

### Experimental Methods

### Antibodies and Reagents

Monoclonal Anti-T7 IgG (Cat #69522-3) was from Novagen. Polyclonal rabbit anti-GFP IgG (Cat. #29779) and Hilyte Plus 647-conjugated goat anti-mouse IgG (Cat #AS-61057-05-H647) were from Anaspec (Fremont, CA). Horseradish-peroxidase (HRP)-conjugated goat anti-rabbit (Cat #111-035-003) or anti-mouse (Cat #115-035-166) IgG were from Jackson ImmunoResearch Laboratories (West Grove, PA). Polyclonal Anti-Rab12 (Cat #18843-1-AP) was from Proteintech (Chicago, IL). Monoclonal Anti-phosphoRab12 (cat #ab256487) was from abcam. Monoclonal Anti-GAPDH (cat #sc-365062) was from Santa-Cruz Biotechnology. Polyclonal anti Ar113b (Cat #177111-1-AP) was from proteintech. Protein A/G PLUS-Agarose (Cat #sc2003) was from Santa Cruz and Glutathione-Agarose (Cat #G4510) and guanosine 5′-[γ-thio] thriphosphate (Cat #G8634) were from Sigma-Aldrich (St. Louis, MO). GSK2578215A (Cat #4629) was from Tocris. Go6976 (Cat #G-1017) was from A.G.Scientific. GF109203X Cat #0741) was from Tocris. LY333531 (Cat #13964) was from Cayman. Rotenone (Cat #ab143145) was from abeam. Lipofedtamine (Cat #11668-027) was from Invitrogen.

### Plasmids Used in the Above Experiments

pEF-T7-RILP, pEF-T7-RILP-L1, pEF-T7-RILP-L2, pEF-T7-RILP(L231A), pEF-T7-RILP(E233A) and pEF-T7-RILP(N235A), pEGFP-C1-Rab12 and pEGFP-C1-Rab12 and pGEX-4T-3-Rab12 were prepared as previously described. pGEX-4T-3-Rab12(K71R) was prepared by site-directed mutagenesis, using the Q5 site-directed mutagenesis kit (NEB, Cat #E0554S) and the following primers: Forward primer: GAGGCCTGCAgGTCCACCGTG (SEQ ID NO: 15), Reverse primer: GCAGAACGTGTCGTCGTG (SEQ ID NO: 16). cDNAs of mouse RILP, RILP-L1, and RILP-L2 were subcloned into the pGEX-4T-3 vector (GE Healthcare, Chicago, IL; named pGEX-4T-3-RILP, pGEX-4T-3-RILP-L1, and pGEX-4T-3-RILP-L2) and pEGFP-C1 vector (Clontech/Takara Bio, Shiga, Japan) and named pEGFP-C1-RILP, pEGFP-C1-RILP-L1, and pEGFP-C1-RILP-L2.

### Cell Culture

### RBL Cells

RBL cells were maintained as adherent cultures in low glucose DMEM, supplemented with 10% FBS, 2 mM L-glutamine, 100 μg/ml streptomycin and 100 units/ml penicillin in a humidified incubator of 5% CO₂ at 37° C.

### BMMCs

Bone marrow-derived cultured mast cells (BMMCs) were isolated from 6 to 10-week-old C57BL/6 mice in complete medium consisting of RPMI 1640 supplemented with 10% FBS (Invitrogen, Carlsbad, CA), glutamine (2 mM), penicillin (100 U/ml), streptomycin (100 mg/ml), pyruvate (1 mM), HEPES (10 mM, pH 7.4), and 2-ME (50 μM). BMMCs were subsequently cultured for 8 weeks in the presence of IL-3 (20 ng/ml; Peprotech, Rocky Hill, NJ). Cell purity (95-97%) was confirmed by analyzing FcεRI and c-kit expression by flow cytometry in addition to testing the functional activity of releasing β-hexosaminidase.

### SH-SY5Y

SH-SY5Y cells were maintained as adherent cultures in high glucose DMEM, supplemented with 15% FBS, 2 mM L-glutamine, 100 μg/ml streptomycin and 100 units/ml penicillin in a humidified incubator of 5% CO₂ at 37° C.

### PC12 Cells

PC12 cells were maintained as adherent cultures in high glucose DMEM, supplemented with 15% FBS, 2 mM L-glutamine, 100 μg/ml streptomycin and 100 units/ml penicillin in a humidified incubator of 5% CO₂ at 37° C.

### Transient Transfection of RBL and PC12 Cells

Transient transfection of RBL cells was performed. Briefly, RBL cells (1.5×10⁷) were transfected with a total of 30-60 μg of cDNAs by electroporation at 300V for 20 msec using an ECM 830 electroporator (BTX, USA). The cells were immediately replated in tissue culture dishes containing growth medium for the desired time periods. PC12 cells (4×10⁴ cells/well) were transiently transfected using lipofectamine 2000.

### Immunostaining and Confocal Analyses

RBL cells (4×10⁵ cells/well) or PC12 cells (4×10⁴ cells/well) were grown on 12-mm round glass coverslips, washed three times with PBS, and fixed for 20 min at room temperature with 4% paraformaldehyde in PBS. Cells were then permeabilized for 20 min at room temperature with 0.1% Triton X-100, 5% FBS, and 2% BSA diluted in PBS. Cells were subsequently incubated for 1 hour at room temperature with the primary Abs, followed by three washes and 1 hour incubation with the appropriate secondary Abs. After washing, the cells were mounted (Golden Bridge Life Science, Mukilteo City, WA) and analyzed using a LEICA SP8 STED high resolution laser scanning confocal microscope (Leica, Wetzlar, Germany) using a 63 oil/1.4 numerical aperture objective.

### Co-Immunoprecipitation Assays

RBL cell lysates (500 μg) prepared in buffer A (50 mM Hepes pH 7.4, 250 mM NaCl, 1 mM MgCl₂, 1% Triton X-100, protease inhibitor mixture, 1 mM PMSF, 2 mM Na₃VO₄) were incubated overnight at 4° C. with either rabbit polyclonal anti-GFP antibodies (2 μg) or mouse monoclonal anti-T7 antibodies (1 μg). Protein A/G-Sepharose (50% v/v) was then added for 1.5 h at 4° C. Immune complexes were collected, washed three times with buffer B (50 mM Hepes pH 7.4, 150 mM NaCl, 1 mM MgCl₂, 0.2% Triton X-100, protease inhibitor mixture, 1 mM PMSF, 2 mM Na₃VO₄), and resuspended in 1× sample buffer, and boiled for 7 min. Proteins were resolved by SDS-PAGE and analyzed by immunoblotting with the desired antibodies.

### Pulldown Assays

Pulldown assays were performed as previously described 2. Briefly, 20 μg of GST fusion proteins or control GST immobilized on Glutathione Agarose beads were incubated for 18 hours at 4° C. with RBL cell lysates (500 μg) prepared in buffer C (50 mM Hepes pH 7.4, 150 mM NaCl, 1 mM MgCl₂, 1% TritonX100, 1 mM PMSF, protease inhibitor mixture, 2 mM Na₃VO₄) in the presence of 0.5 mM GTPγS. At the end of the incubation period, beads were sedimented by centrifugation at 5000×g for 5 minutes at 4° C., washed 4 times in buffer C with 0.2% TritonX100, and finally suspended in sample buffer, boiled for 7 minutes, and subjected to SDS-PAGE and immunoblotting. In pulldown experiments of endogenous phosphoRab12, cell lysates were prepared in buffer D (50 mM Hepes pH 7.4, 150 mM NaCl, 1 mM MgCl₂, 1% TritonX100, 1 mM PMSF, protease inhibitor mixture, 2 mM Na₃VO₄, 10 mM NaPPi and 80 mM β-glycerophosphate) and the pulldown assay was conducted in the absence of GTPγS. Beads were washed in buffer D with 0.2% TritonX100.

### Western Blot Analysis

Samples were separated by SDS-PAGE using 10-12% polyacrylamide gels and electrophoretically transferred to nitrocellulose membranes. Blots were blocked for 20 min in TBST (10 mM Tris-HCl, pH 8.0, 150 mM NaCl, and 0.05% Tween 20) containing 5% skim-milk, followed by overnight incubation at 4° C. with the desired primary Abs. Blots were washed three times and incubated for 1 hour at room temperature with HRP-conjugated secondary Ab. Immunoreactive bands were visualized by the ECL method according to standard procedures. The intensity of the immunoreactive bands was quantified using ImageJ software.

### Molecular Dynamics

The GDP bound conformation of Rab12 was modeled using swiss model with Rab12 X-RAY structure (PDB 21L1) as a template. Missing loop coordinates (residues 64-77) was completed using Ypt1, RABGTPase from yeast (PDB 2BC6) as a template. The GTP bound conformation was modeled using HHPRED and Modeller with Rab7 X-RAY structure (PDB 1YHN) as a template. Reconstructing RILP dimer was done using the crystal symmetry of RILP structure bound to Rab7 (PDB 1YHN) with Pymol. Docking RILP dimer to Rab12 models was done using GRAMM-X and Patchdock followed by the refinement docking tools Firedock and ZDOCK²⁸. MD simulation was conducted for 162 nanoseconds.

### Activation of RBL Cells or BMMCs

Cells were incubated overnight with a 1 to 512 dilution of conditioned medium derived from a hybridoma secreting DNP specific IgE. After three washes in Tyrode's buffer (20 mM Hepes pH 7.4, 137 mM NaCl, 2.7 mM KCl, 1.8 mM CaCl₂), 1 mM MgCl₂, 0.4 mM NaH₂PO₄, 5.6 mM glucose, and 0.1% BSA), cells were stimulated in the same buffer for 30 minutes at 37° C. with the desired stimuli [i.e. a combination of 1 μM 4-bromo-calcium ionophore A23187 (Ion) and 50 nM of the phorbol ester (TPA), or 50 ng/ml DNP-HSA (Ag)]. Samples were subsequently lysed for 30 minutes in lysis buffer D (50 mM Hepes pH 7.4, 150 mM NaCl, 1 mM MgCl₂, 1% Triton X-100, protease inhibitor mixture, 1 mM PMSF, 2 mM Na₃VO₄, 10 mM NaPPi and 80 mM β-glycerophosphate) and cell lysates analyzed by western blotting.

### Rab12 Phosphorylation Analyses

Cells (RBL, BMMCs or SH-SY5Y) were grown overnight in growth medium or medium containing 400 nM TPA, where indicated. Next day cells were washed three times with Tyrode's buffer and either left untreated or pre-incubated with the desired inhibitor [i.e. 1 μM Go6976, 1 μM MRT68921, 2 mM EGTA, 10 μM GSK2578215A, 1 μM LY333531] for 30 minutes. Cells were then either left untreated or stimulated with a combination of 1 μM 4-bromo-calcium ionophore A23187 (Ion) and 50 nM of the phorbol ester (TPA), in the absence or presence of inhibitor, for additional 30 minutes. Cells were then washed with PBS and lysed for 30 minutes in lysis buffer D. Cell lysates were analyzed by western blotting. For Rab12 phosphorylation in PC12 cells, cells were grown for 48 hours either in growth medium or in medium supplemented with 1 μM LY333531 or 10 μM GSK2578215A in the absence or presence of 100 nM rotenone. For Ion/TPA-stimulated phosphorylation, cells grown in medium only or medium containing inhibitors, were stimulated with 1 μM 4-bromo-calcium ionophore A23187 (Ion) and 50 nM of the phorbol ester (TPA) for 30 minutes. Cells were processed as above.

### Inhibitors:

Go6976: PKC inhibitor. selectively inhibits PKCα and PKCβ1.
GSK2578215A: LRRK2 inhibitor.
MRT68921: ULK1/2 inhibitor.
EGTA: Ca²⁺-chelating agent.
LY333531: PKCβ inhibitor

### Statistical Analysis

Data are expressed as means±SEM. The P values were determined by an unpaired two-tailed Student's t test or by ANOVA followed by Bonferroni corrected post-hoc t-test, for multiple comparisons.

### EXPERIMENTAL RESULTS

### Example 1

The RILP Family Members, RILP, RILP-L1 and RILP-L2, Form Homodimers, but do not Heterodimerize with Each Other

Though Rab12 was shown to interact with its RILP family members in pulldown assays, yeast two hybrid screening identified only Rab12 binding to RILP-L1. This discrepancy prompted the inventors to explore the possibility that RILP or RILP-L2 may interact with Rab12 by forming a heterodimer with RILP-L1. The inventors relied on the observation that RILP was shown to form homodimers, and therefore asked whether the other members of the family can form homo- or heterodimers. To this purpose, they examined the capacity of GFP-fused versions of RILP, RILP-L1 and RILP-L2 to co-immunoprecipitate T7-tagged versions of themselves or the other members of this family, that were co-transfected in RBL cells, a model mast cell line. The results of these experiments confirmed the ability of RILP to form homodimers or homo complexes (FIG. 1A). Further, they have also demonstrated the ability of GFP-RILP-L1 to co-immunoprecipitate T7-RILP-L1 and the ability of GFP-RILP-L2 to co-immunoprecipitate T7-RILP-L2 (FIG. 1A). Therefore, like RILP, also the two other members of this family, RILP-L1 and RILP-L2 can homodimerize. In sharp contrast, none of the RILP family members was able to co-immunoprecipitate any of the other members. Hence, immunoprecipitated GFP-RILP failed to co-immunoprecipitate with T7-RILP-L1 or T7-RILP-L2, and neither did GFP-RILP-L1 co-immunoprecipitate with T7-RILP-L2 (FIG. 1B). Similar results were obtained in the reciprocal experiments, where the ability of T7-tagged RILP, RILP-L1 or RILP-L2, to co-immunoprecipitate their co-transfected GFP-fused homologs was tested (FIGS. 1C, 1D). Since neither one of the RILP family members demonstrated any heterodimerization activity, these results imply that RILP family members interact with Rab12 independently of each other.

### Example 2

### Lysine 71 is Critical for Rab12 Binding of RILP-L1 and RILP-L2, but is Dispensable for Binding of RILP

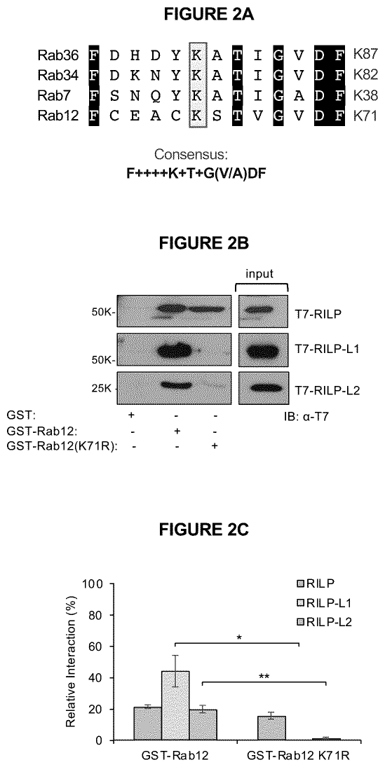

Aiming to delineate the binding site of Rab12 for its RILP family effectors, the inventors were based on studies, which identified lysine 38 and 82 in Rab7 and Rab34, as critical for their interactions with RILP. Sequence alignment of the amino acids that are proximal to those lysine residues, prompted us to propose the amino acid sequence F++++K+T+G(V/A)DF, that is also present in Rab36, another RILP-interacting protein, and in Rab12 (FIG. 2A), as a consensus for RILP binding. Therefore, lysine 71, the corresponding lysine in Rab12, was substituted to arginine, that preserves the positive charge of the amino acid, but may interfere with its specific functions, and examined the impact of this mutation on Rab12 pulldown efficacy. In contrast to the inventors' expectation, GST-Rab12(K71R) retained its capacity to pull down T7-tagged RILP from RBL cell lysates (FIGS. 2B, 2C). However, this mutation significantly inhibited the ability of Rab12 to pull down either RILP-L1 or RILP-L2 (FIGS. 2B, 2C). Therefore, while these results support the positioning of K-71 at Rab12 binding site of RILP-L1 and RILP-L2, they imply that Rab12 binding site of RILP might either be distinct or redundant.

### Example 3

Molecular Dynamics Simulations of the Rab12-RILP Complex Predict a Ternary Complex that Involves Two Interfaces within Rab12 and the RILP Homology Domain (RHD)

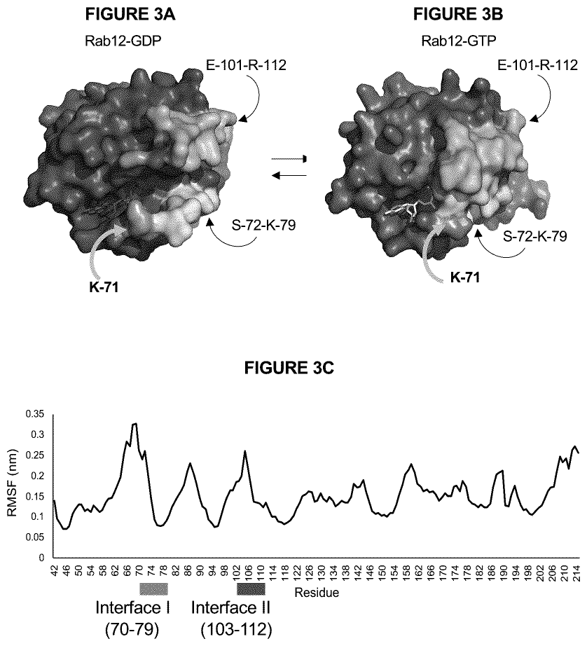

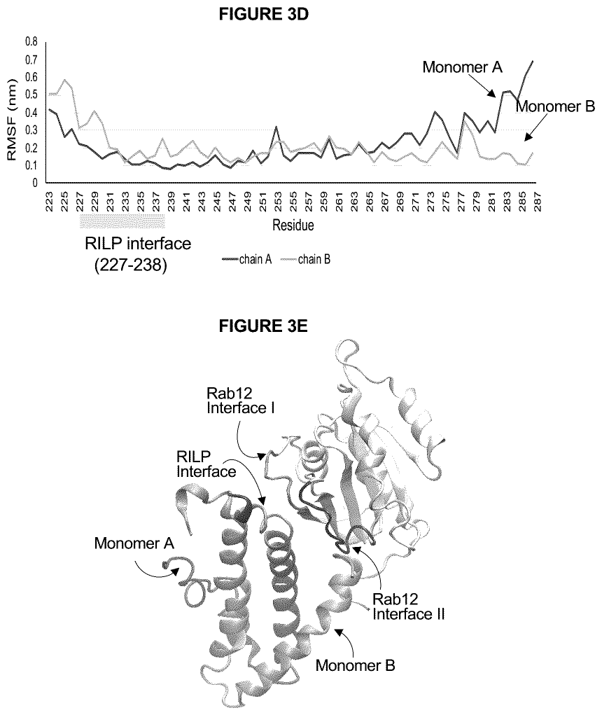

In an alternative approach to understand the molecular dynamics of the Rab12-RILP complex and in particular the positioning of K-71 in this context, a computational model of the Rab12-RILP complex was generated. GDP-bound conformation of Rab12 using Rab12 X-ray structure (PDB 21L1) as template was modelled, and the GTP-bound conformation on the basis of Rab7 structure (PDB 1YHN), relying on the fact that the root-mean-square deviation (RMSD) between the atoms of superimposed Rab7 and Rab12 is 0.743 \acute{Å}, suggesting that their structures are reasonably similar. Based on these models (FIGS. 3A, 3B), Rab12 activation is associated with a conformational shift in loops comprising amino acids serine 72 to lysine 79 and glutamic 101 to the arginine at position 112 (FIGS. 3A, 3B), as is reflected in the change in distance between V-74 to F-103, from 14.3 Å in the GDP-bound conformation of Rab12 to 9 Å in its GTP-bound, active conformation, creating a pocket involving the arginine residue at position 50 (FIGS. 3A, 3B). The active Rab12 model was docketed to a RILP homodimer, on the basis of the published structure of the Rab7-RILP dimer complex, and subjected the complex to molecular dynamics (MD) simulations, to predict the modes of Rab12-RILP interactions at atomic resolution. Analyzing the root-mean-square fluctuations (RMSF) of each protein during simulations predicted the existence of two interfaces between Rab12 and the RILP dimer (FIGS. 3C, 3D). The first interface spanned amino acids C-70 to K-79, which include a predicted binding site of Rab12 for its effectors (FIGS. 3C, 3E). The second interface spanned amino acids F-103 to R-112 (FIGS. 3C, 3E), which together with the first interface, are predicted by the model to change location during the Rab12 activation cycle and are therefore expected to interact with RILP in a GTP dependent fashion (FIG. 3A). Restricted mobility was also noted for Rab12 residues L-42 to I-46 (FIG. 3C), however as these residues reside within the guanine nucleotide binding site, their mobility is likely to be restricted by the binding of GTP, when comparing to the unbound protein. RILP contains two coiled-coil (CC2) domains, of which the CC2 domain present within its C-terminal half, is conserved within all three members of this family (i.e. the RILP Homology Domain, RHD). This domain was shown to mediate RILP binding to Rab7, Rab34 and Rab36. Consistent with the involvement of the RHD in mediating RILP interactions with Rab GTPases, the model has positioned residues L-227 to K-238, which comprise the RILP RHD, at the Rab12 interface (FIGS. 3D, 3E). In fact, both interfaces of Rab12 were predicted to interact with the RHD of same RILP monomer, while an additional contact was predicted to form between Rab12 and the second RILP monomer (FIGS. 3D, 3E). Therefore, unlike the Rab7-RILP tetrameric complex, that consists of a RILP homodimer complexed to two molecules of Rab7, Rab12 is predicted to form a ternary complex consisting of a RILP homodimer and a single molecule of Rab12.

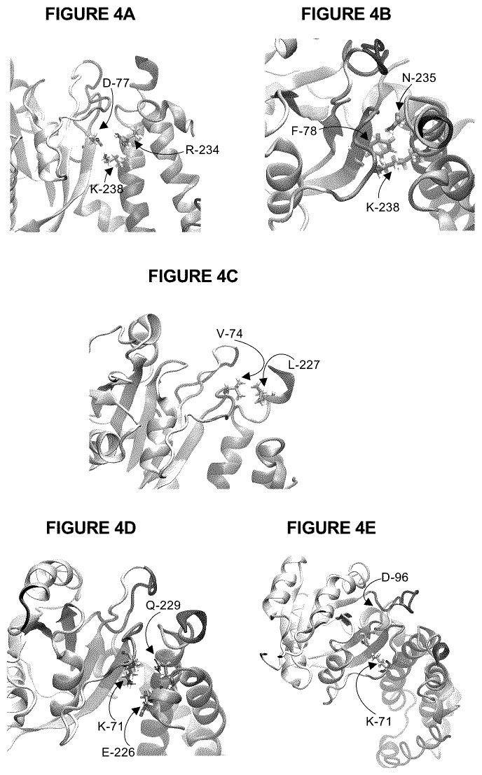

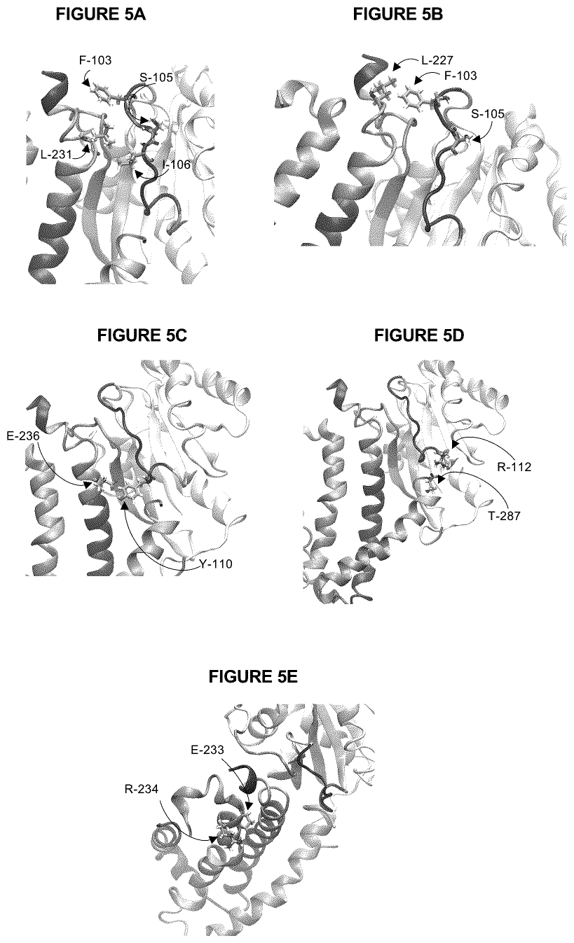

Further analysis of the MD trajectories predicted stable interactions between D-77 that resides in the first interface of the Rab12-RILP complex, and residues R-234 and K-238 of a single RILP monomer (Table 1 and FIG. 4A), phenocopying the interaction of Rab7 D-44, the equivalent of Rab12 D-77 in Rab7 (FIG. 2a), with residues R-255 and K-259, the equivalents of mouse R-234 and K-238 in human RILP. MD trajectories also predicted a highly stable interaction between F-78 and RILP residue K-238 and a more labile interaction between this residue and RILP N-235 (Table 1 and FIG. 4B), in analogy to the interactions of F-45, the Rab7 equivalent of Rab12 F-78 (FIG. 2A), with N-256 and K-259 residues in human RILP residues. Interestingly, though 1-41 of Rab7 is replaced in Rab12 by V-74 (FIG. 2A), and F-248, which in human RILP interacts with 1-41, is replaced in mouse RILP by L-227, the MD simulations predicted an analogous stable interaction between Rab12 V-74 and RILP-L227 (Table 1 and FIG. 4C). Contradictory however, to the Rab7-RILP complex, in which Rab7 K-38 plays an important role via its interactions with E-247 and Q-250 in human RILP, no interactions were predicted between Rab12 K-71, the equivalent of the Rab7 K-38 residue, and E-226 and Q-229, the equivalent residues in mouse RILP (Table 1 and FIG. 4D). In fact, K-71 seemed to be engaged in an intramolecular interaction mediated by a hydrogen bond with D-96 (Table 1 and FIG. 4E), thus providing an explanation for the lack of impact of the K-71 mutation on the binding of RILP.
In sharp contrast to the interactions within the first interface of the Rab12-RILP complex, which largely recapitulated the interactions of the first interface of the Rab7-RILP complex, the second interface of the Rab12-RILP complex is unique, sharing no homology with the Rab7-RILP complex. MD trajectories predicted interactions between both F-103 and 1-106 of Rab12 and same RILP residue L-231. Hence, during 52% of time of simulation, L-231 was located in close proximity to F-103, while during 41% of time, L-231 was proximal to 1-106 (Table 1, FIG. 5A). A short-lived interaction, accounting for only 7% of time of simulation, was recorded between L-231 and S-105 of Rab12 (Table 1, FIG. 5A). Intriguingly, this amino acid is the site of Rab12 phosphorylation by the Parkinson's disease-related kinase Leucine-Rich Repeat kinase 2 (LRRK2), which stimulates Rab12 binding of RILP-L2, but not of RILP-L1. Whether or not LRRK2-mediated phosphorylation of Rab12 affects binding of RILP is presently unknown.

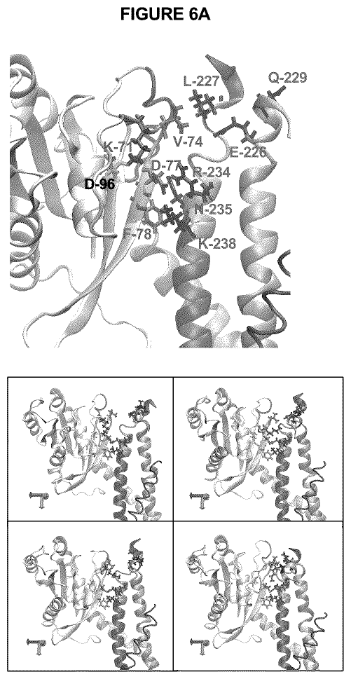

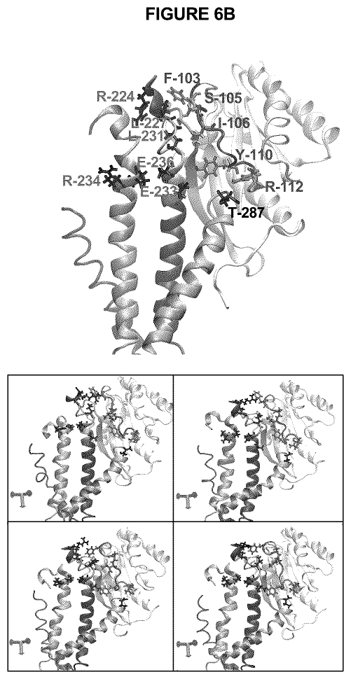

MD trajectories also disclosed interactions between F-103 and L-227. Thus, at time of simulation that F-103 was not in contact with L-231, this residue was engaged in an interaction with L-227, which also forms contact with V-74 of the first Rab12 interface (Table 1, FIG. 5B). Two additional interactions of the second interface of Rab12 were predicted for Y-110 and RILP residue E-236 (Table 1, FIG. 5C), and R-112 with RILP residue T-287 (Table 1, FIG. 5D). However, unlike residues F-103, 1-106 and Y-110, which form contacts with same RILP monomer (Monomer A, FIG. 5D), R-112 forms a stable hydrogen bond with the threonine residue of the second RILP monomer (Monomer B, FIG. 5D), consistent with the RMSF variability of the C-terminal regions of the two RILP monomers (FIG. 3D). Finally, a strong and stable interaction was predicted between E-233 of monomer A and R-234 of monomer B (Table 1, FIG. 5E), implicating these residues in RILP dimerization. Taken together, the simulated model suggests a ternary Rab12-RILP homodimer complex, governed by the RHD of one RILP monomer that associates with two interfaces of Rab12, of which the second interface also associates with the second monomer of the RILP dimer (FIG. 6).

Table 1 describing: Rab12 (mouse) and RILP (mouse) contacts along the MD trajectories. The table presents the type of bonds that are generated between atoms within Rab12 RILP monomer atoms. The percentage of time that the contacts are maintained along the trajectory are indicated.

TABLE 1

| | | | Dis- | | Time of |
|---|---|---|---|---|---|
| | | | tance | Mode of | simulation |
| Rab12 | RILP(A) | RILP(B) | (nm) | interaction | (%) |
| K-71 (NZ) | E-226 | — | | unstable | |
| | (OE1/OE2) | | | | |
| K-71 (NZ) | Q-229 | — | | unstable | |
| | (NH1/NH2) | | | | |
| K-71 (NZ) | — | — | <0.3 | Hydrogen | 59.7 |
| D-96 | | | | bond | |
| (OD1/OD2) | | | | | |
| V-74 (CG) | L-227 (CD) | — | 0.3 | Van Der | 60 |
| | | | | Waals | |
| D-77 | K-238 (NZ) | — | <0.35 | Strong salt | 99 |
| (OD1/OD2) | | | | bridge | |
| D-77 | R-234 | — | <0.5 | Medium | 96 |
| (OD1/OD2) | (NH1/NH2) | | | strength | |
| | | | | salt bridge | |
| F-78 | N-235 | — | | No | |
| | (ND2) | | | interaction | |
| F-78 (CG) | K-238 (NZ) | — | | Stable | 99.8 |
| | | | | cation-pi | |
| | | | | interaction | |
| F-103 (CG) | L-227 | — | 0.33 | Van Der | 60 |
| | (CD1/CD1) | | | Waals | |
| F-103 | R-224 | — | | No | |
| | | | | interaction | |
| F-103 | L-231 | — | 0.3 | Van Der | 52.4 |
| (phenyl ring) | (CD1/CD2) | | | Waals | |
| S-105 | L-227 | — | | No | |
| | | | | interaction | |
| S-105 (OG) | L-231 (O/N) | — | | No | |
| | | | | interaction | |
| I-106 (CD) | L-231 | — | 0.32 | Van Der | 41 |
| | (CD1/CD2) | | | Waals | |
| Y-110 (OH) | E-236 | — | <0.3 | Hydrogen | 56.5 |
| | (OE1/OE2) | | | bond | |
| R-112 | — | T-287 | <0.3 | Hydrogen | 89 |
| (NH1/NH2) | | (OD1/OD2) | | bond | |
| — | E-233 | R-234 | <0.3 | Strong salt | 97.7 |
| | (OE1/OE1) | (NH1/NH2) | | bridge | |

### Example 4

### Mutational Analysis Supports the Involvement of RILP RHD in Rab12 Binding

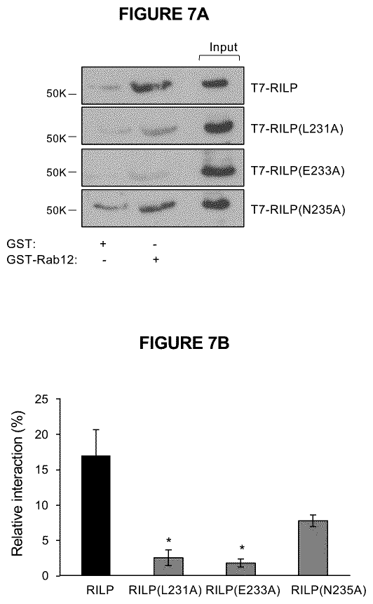

To substantiate the involvement of RILP RHD in binding of Rab12, a mutational analysis of RILP RHD was performed, focusing on L-231, which based on the model forms contact with both F-103 and I-106 of Rab12, and whose mutation impaired RILP interactions with Rab7, Rab34 and Rab36. Indeed, alanine substitution of this residue has completely abrogated RILP pulldown by immobilized Rab12 (FIG. 7). Also, the impact of a mutation in N-235, whose alanine substitution impaired binding to Rab34 was analyzed, but did not affect binding to Rab7 or Rab36. In accordance with the model, which predicted no stable interaction between this RILP residue and Rab12 (Table 1), this mutant showed reduced affinity to Rab12, though binding was not significantly different (FIG. 7). Finally, the impact of alanine substitution of E-233 was investigated, whose alanine replacement was shown to prevent RILP binding to Rab7 and Rab34, but not binding to Rab36, and which based on the model does not directly interact with Rab12, but plays a role in RILP dimerization. These experiments demonstrated that alanine substitution of E-233 significantly inhibited RILP pulldown by Rab12 (FIG. 7), thus indicating the importance of RILP dimerization for Rab12 interaction, consistent with the predicted model of a ternary complex consisting of Rab12 and a homodimer of RILP.

### Example 5

RILP RHD Mutants have Different Impacts on the SG Distribution in MCs

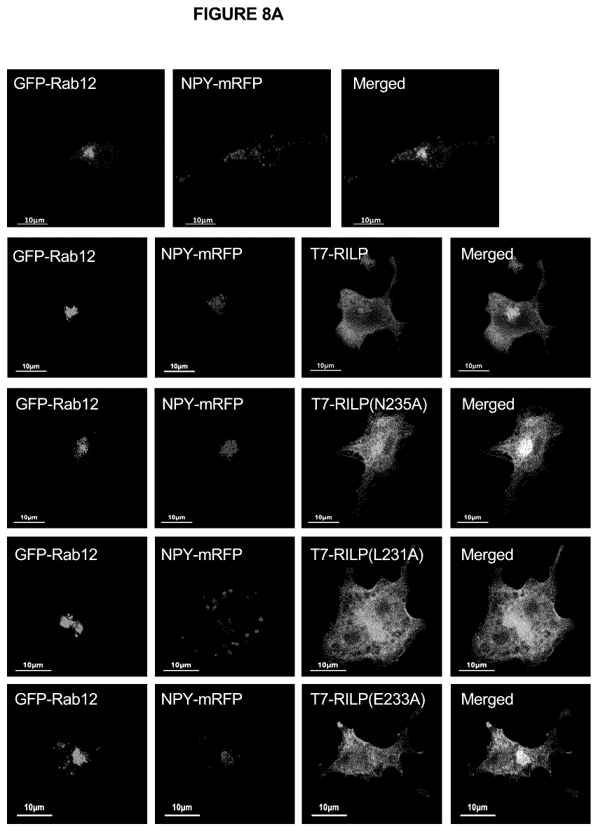

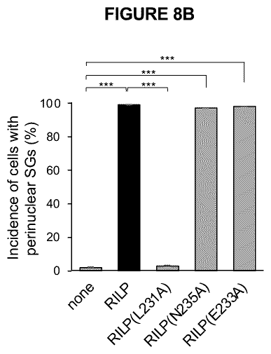

To investigate the functional consequences of impairment of Rab12-RILP interaction, the influence of overexpression of RILP RHD mutants on the cellular distribution of the SGs in MCs was investigated. For this purpose, RBL cells that were co-transfected with RILP or RILP RHD mutants and NPY-mRFP, were visualized by confocal microscopy which have previously shown to serve as a genuine SG reporter. In agreement with previous results, in control cells, NPY-mRFP-labeled SGs distributed throughout the cell, while Rab12 localized to the perinuclear region (FIG. 8), which have previously identified as the ERC. Also consistent with previous results, the SGs localized to the perinuclear region in 99% of cells that co-expressed Rab12 and RILP (FIG. 8). RILP(N235A), the RILP mutant that is capable of binding Rab12, replicated the perinuclear accumulation of the SGs (FIG. 8). In sharp contrast, the SGs remained scattered in 97% of cells that overexpressed the RILP(L231A) mutant, that does not bind Rab12 (FIG. 8). Surprisingly, RILP(E233A), which in a similar way to RILP(L231A), has lost its ability to bind Rab12, successfully enforced perinuclear clustering of the SGs (FIG. 8). These results therefore suggest that a protein, other than Rab12, may intervene between the dynein-RILP complex and the SGs to drive their minus end transport. Such protein may either function redundantly with Rab12, or compensate the absence of Rab12-RILP interaction.

### Example 6

Perinuclear Targeting of Rab12 does not Depend on Rab12 Interactions with its RILP Family Effectors

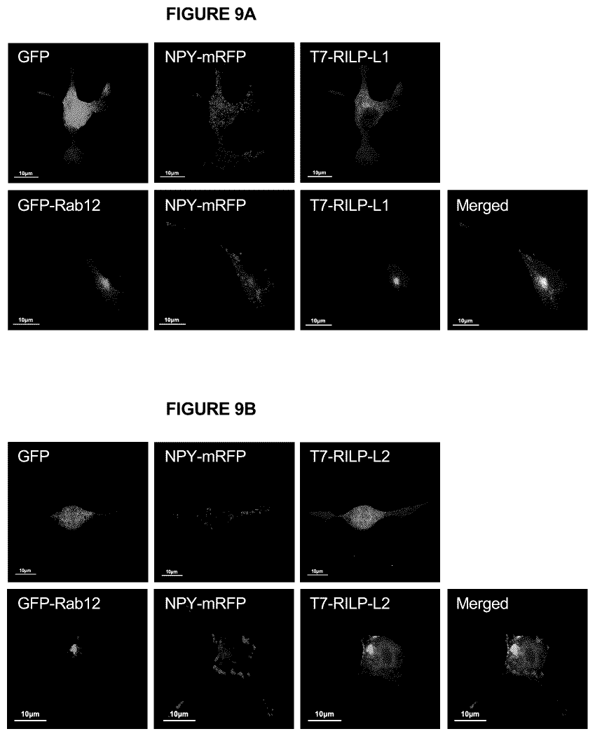

The inventors noticed that co-expression of Rab12 with the non-interacting RHD mutants of RILP, RILP(L231A) or RILP (E233A), did not affect the cellular location of overexpressed Rab12, which remained perinuclear (FIG. 8). These results suggested that RILP plays no role in Rab12 targeting to the ERC. Therefore, it was questioned whether RILP-L1 or RILP-L2, the other Rab12 effectors, played such role. However, while both RILP-L1 and RILP-L2 localized to the cytosol when overexpressed alone in the RBL cells, these effectors redistributed from the cytosol to the perinuclear region when co-expressed with Rab12 (FIG. 9). Therefore, these results confirmed the interactions of Rab12 with these effectors, demonstrating their occurrence also in intact cells, but they also implicated Rab12 in the targeting of RILP-L1 and RILP-L2 to the ERC, rather than the other way around. Therefore, Rab12 acquires its ERC location independently of its interactions with RILP family members, and may play a role in their cellular targeting. Notably, co-expression of Rab12 with neither RILP-L1 nor with RILP-L2 had any impact on the cellular distribution of the SGs (FIG. 9), consistent with their lack of a dynein binding domain.

### Example 7

### Rab12 is Phosphorylated in Activated Mast Cells

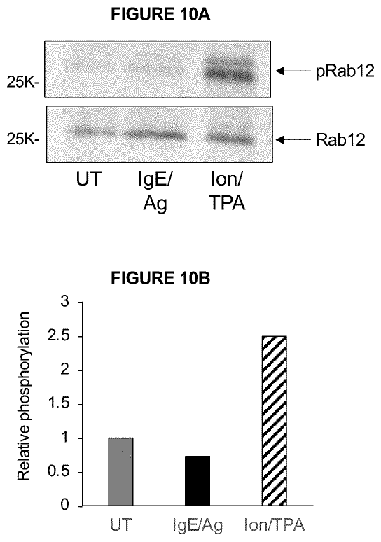

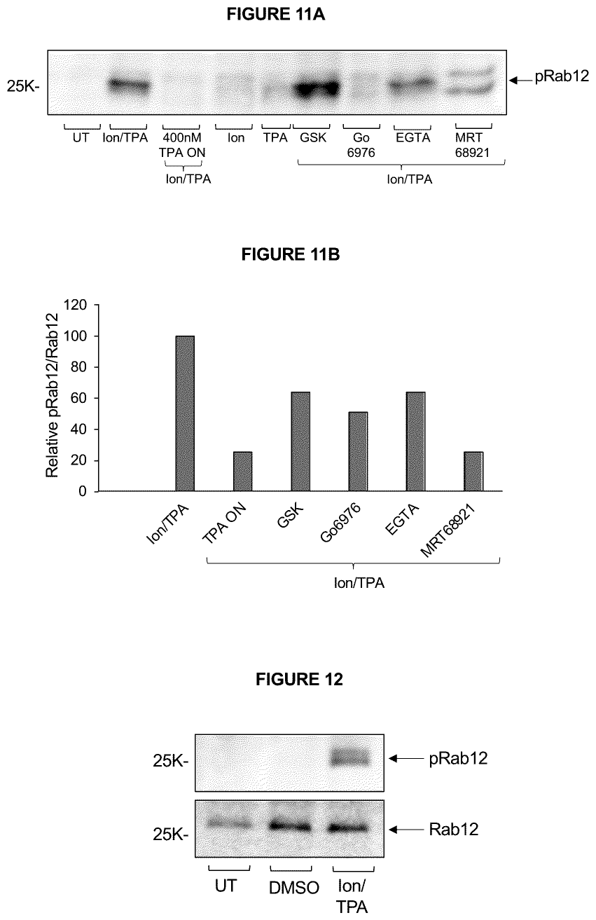

Examining the status of Rab12 phosphorylation in RBL cells, a mast cell line, widely used as model, under basal and triggered conditions, revealed a faint signal in resting cells, which was slightly (by −25%) reduced in cells that were triggered by IgE/antigen (IgE/Ag) (FIG. 10). This phosphorylation was increased by 2.5-fold in response to a combination of Ca′ ionophore and the phorbol ester TPA (Ion/TPA), that triggers MC degranulation, by acting downstream of the FcεRI (FIG. 10). These experiments were repeated using in vitro differentiated, bone marrow derived MCs (BMMCs) and obtained similar results (FIG. 11).

### Example 8

### Protein Kinase C and Ulk1/2 are Involved in Rab12 Phosphorylation in Activated MCs

Given the identification of Rab12 as a physiological substrate of LRRK2, next it was investigated whether MCs endogenously express this kinase. Surprisingly, PCR analysis demonstrated that while RBL cells, do indeed express this kinase, the less mature BMMCs do not express endogenously LRRK2, therefore implicating a kinase different than LRRK2 in Rab12 phosphorylation in BMMCs. To identify this kinase the cells were subjected to treatment with a panel of known kinase inhibitors, and the sensitivity of Rab12 phosphorylation to such treatments was analyzed. Specifically, GSK2578215A, an inhibitor of LRRK2, Go6976 an inhibitor of classical, Ca²⁺-dependent PKCs, the Ca²⁺ chelator EGTA and MRT68921, an inhibitor of the Ulk1/2 kinases were included. In particular, the latter inhibitor was included because Ulk1/2 was shown to phosphorylate the Rab12 GEF protein, Dennd3. Results demonstrated that Ion/TPA-stimulated phosphorylation of Rab12 was significantly inhibited by either Go6976 or MRT68921, implicating PKC and Ulk1/2 in stimulating Rab12 phosphorylation (FIG. 11).

### Example 9

### Rab12 Phosphorylation in SH-SY5Y

Rab12 phosphorylation was also tested in SH-SY5Y cells, a human neuroblastoma cell line often used as model for neuronal cells. Results demonstrated that same as in MCs, phosphorylation of Rab12 can be effectively induced by a combination of Ion/TPA (FIG. 12), therefore indicating that Rab12 phosphorylation by kinases other than LRRK2 may also occur in other cell types, including neuronal cells.

### Example 10

### Rab12 is Phosphorylated in a PD Model

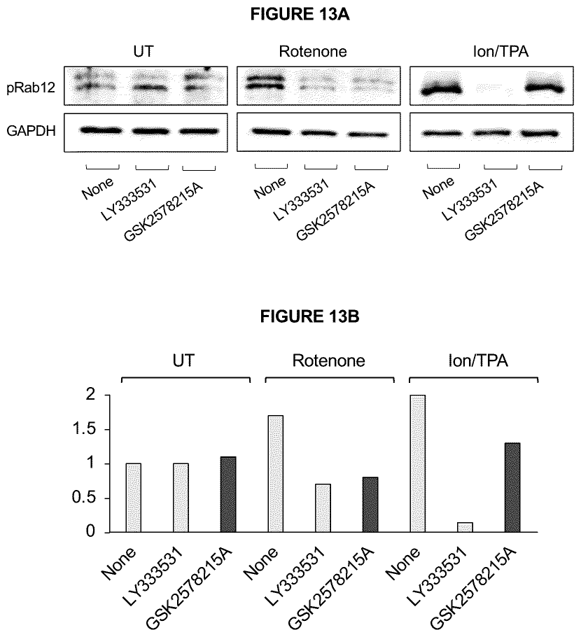

To investigate Rab12 phosphorylation in relation to PD, we used the PC12 cells as a model of dopamine neurons and have subjected the cells to rotenone, relying on the known connection between rotenone-induced oxidative stress and dopaminergic neuron degeneration. As a positive control, cells were also exposed to Ion/TPA and the level of Rab12 phosphorylation was analyzed. Same as in MCs or SH-SY5Y cells, the combination of Ion/TPA has induced phosphorylation of Rab12 in PC12 cells (FIG. 13). This phosphorylation was resistant to GSK2578215A, but could be completely inhibited by LY333531, a selective inhibitor of the beta isoform of PKC (FIG. 13). Rab12 phosphorylation was also induced in rotenone-treated cells, supporting an association between Rab12 phosphorylation and PD (FIG. 13). Strikingly, this phosphorylation was significantly inhibited by either GSK2578215A or LY333531 (FIG. 13). These results have therefore demonstrated a hitherto unknown connection between LRRK2 and PKCβ in rotenone-activated PC12 cells. In this context, it is interesting to note that PKCβ is activated by oxidative stress, which is also an important player in aging, a dominant factor in PD.

### Example 11

### Rab12 Phosphorylation has Different Impacts on Effector Binding by Rab12

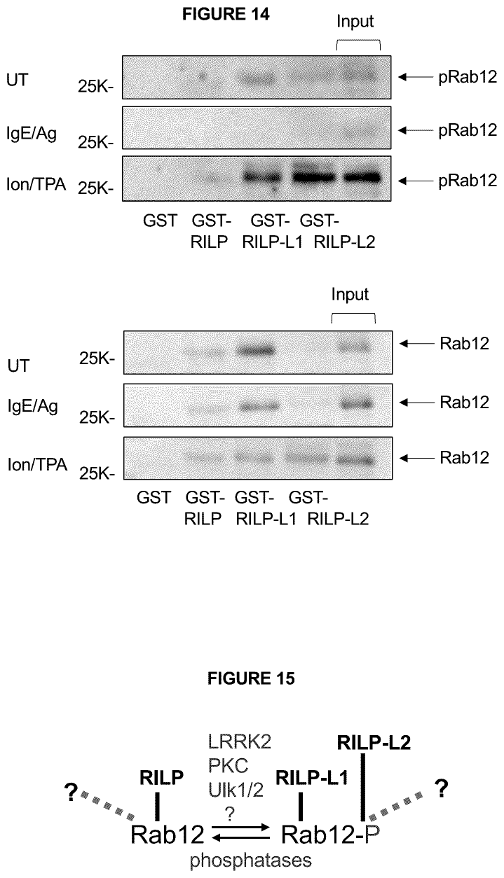

Next, it was assessed whether Rab12 has any impact on Rab12 interactions with its RILP family effectors. To this end, lysates derived from resting RBL cells or RBL cells that were triggered with either IgE/Ag or Ion/TPA, were subjected to pulldown assays by chimeric proteins consisting of GST-fused to either RILP, RILP-L1, or RILP-L2, or control GST. Except for control GST, all three GST fusion proteins pulled down endogenous Rab12 from cell lysates derived from resting RBL cells, however, GST-RILP-L1 was by 3-fold more effective than GST-RILP and by 9-fold more effective than GST-RILP-L2 (FIG. 14). Therefore, under these conditions, the efficacy of Rab12-effector interactions was RILP-L1>RILP>>>RILP-L2. Results were essentially the same for the pulldown from lysates derived from IgE/Ag-triggered cells, though some reduction was noted in Rab12 pulldown by GST-RILP-L1 (FIG. 14). However, subjecting the cells to an Ion/TPA trigger resulted in a 4-fold increase in Rab12 pulldown by GST-RILP-L2 (FIG. 14). Strikingly, analyzing the phosphorylation status of the effector-bound Rab12 (i.e. pulled down Rab12) revealed a mirror image. Hence, while hardly any phosphorylated Rab12 was detected in the GST-RILP pulldown, both GST-RILP-L1 and GST-RILP-L2 have clearly pulled down phosphorylated Rab12 (FIG. 14). This change was even more prominent in Rab12 pulldowns from Ion/TPA-triggered cells, where GST-RILP-L2 was the most effective interactor, followed by GST-RILP-L1, while no phosphorylated Rab12 could be detected in the GST-RILP pulldown (FIG. 14). Thus, the efficacy of phosphoRab12-effector interactions was RILP-L2>RILP-L1>>>RILP. Therefore, these results clearly demonstrated a distinct preference of Rab12 RILP family effectors, whereby RILP-L1 and even more so RILP-L2 favor the phosphorylated form of Rab12, while RILP clearly prefers its non-phosphorylated form (FIG. 15).

Taken together the above results suggest that under basal conditions a fraction of Rab12 is phosphorylated. The finding that this basal phosphorylation of Rab12 occurs only in RBL cells and not in BMMCs, suggests that it is mediated by LRRK2. Under these conditions Rab12 primarily interacts with RILP and also with RILP-L1, whereas the interaction with RILP-L2 is minimal, and confined to the phosphorylated fraction of Rab12. When the cells are activated by an IgE/antigen (i.e. IgE/allergen) trigger, Rab12 phosphorylation is decreased, most likely by the activation of a yet unknown phosphatase. This leads to an increase in Rab12 binding of RILP and therefore to a negative control of MC degranulation. A reduction in binding to RILP-L1 is also observed. When the cells are activated by a combination of a Ca²⁺ ionophore and the phorbol ester TPA, conditions that are known to activate classical protein kinase C (PKC), Rab12 is phosphorylated and the sensitivity of this phosphorylation to inhibitors of PKC and Ulk1/2 implicates these kinases in this phosphorylation. Phosphorylation of Rab12 then enhances its interaction with RILP-L1 and even more so with RILP-L2, while RILP does not bind any of the phosphorylated Rab12. This finding implies that during their activation by Ion/TPA, and unlike their activation by IgE/Ag, MCs are not subjected to negative regulation by Rab12-RILP-mediated retrograde transport of their SGs, which explains why MCs release their SG mediators much more extensively when triggered by Ion/TPA as compared to IgE/Ag. In addition, actin rearrangements, MyoVa dependent anterograde transport and formation of membrane protrusions that might occur following complex formation between phopshoRab12 and RILP-L2, may further stimulate MC degranulation.

Furthermore, the fact that the antibodies used to monitor Rab12 phosphorylation by either LRRK2 or PKCβ are directed against same serine 106 in human Rab12, implies that the same serine residue is phosphorylated by either LRRK2 or PKCβ. Therefore, phosphorylation by either kinase inflicts similar consequences, i.e. enhanced affinity to binding of RILP-L1 and RILP-L2, but not RILP. These results strongly imply that Rab12 phosphorylation maintains the homoeostatic balance of Rab12 responses by controlling its distribution between RILP and RILP-L1/2 (FIG. 15). Therefore, hyperactivation of either LRRK2, as is the case in PD, or PKCβ, that lead to hyperphosphorylation of Rab12, impair Rab12 balanced interactions by favouring its interactions with RILPL1/2. The resulting disturbance of the cell homoeostasis then contributes to pathogenesis.

### Example 12

Rab12 Interaction with RILP

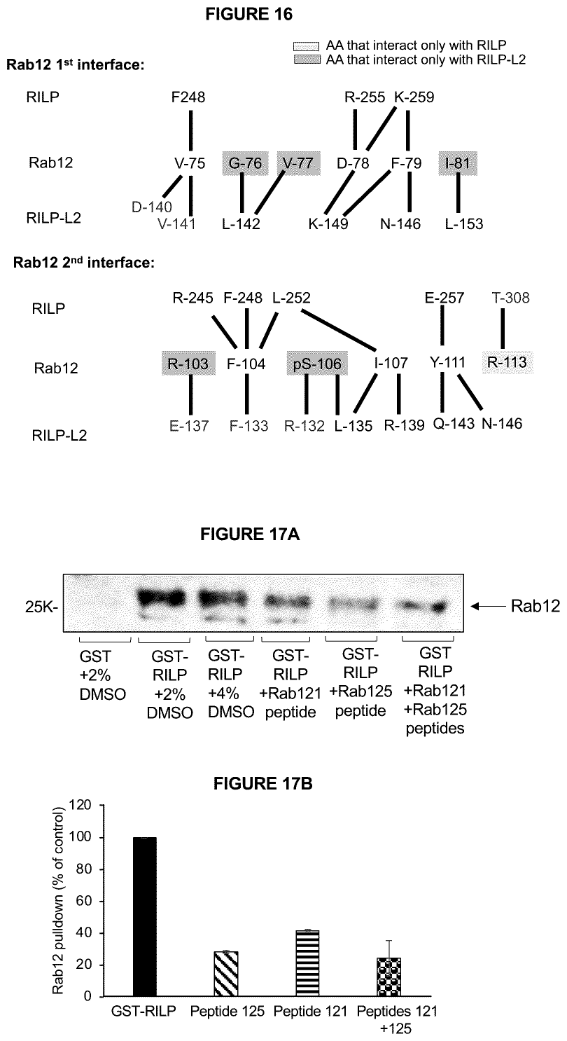

Based on above hypothesis, peptides, or molecules that mimic their function, that are designed to restore the balance in Rab12 interactions, would in re-establish the cellular homeostasis and arresting pathologies that result from such imbalance, such as in Parkinson's disease or any other disease linked with imbalance of Rab12-effector interactions. Towards the rational design of such peptides, in silico modelling and molecular dynamics were combined with mutational analyses to map the binding sites of Rab12 for RILP and RILP-L2. The results show that Rab12 interacts, respectively with RILP and RILP-L2, via two interfaces in Rab12 (FIG. 16). The results also demonstrate that some amino acids within these interfaces participate in the binding of both RILP and RILP-L2, whereas others are specific for either the binding of RILP or RILP-L2 (FIG. 16).

Five (5) peptides that comprise the sequences in human Rab12 and modification thereof were synthesized:

69-83
(SEQ ID NO: 8)
EACKSTVGVDFKIKT (peptide Rab 125);
102-116
(SEQ ID NO: 4)
ERFNSITSAYYRSAK (peptide Rab 121);

And modified versions of peptide 121, where serine 106 was substituted by aspartate:

(SEQ ID NO: 5)
ERFNDITSAYYRSAK (peptide Rab 122);

Or serine 114 by aspartate

(SEQ ID NO: 6)
ERFNSITSAYYRDAK (peptide Rab 123);

Or both:

(SEQ ID NO: 7)
ERFNDITSAYYRDAK (peptide Rab 124).

Testing some of these peptides in pulldown assays revealed that peptides Rab121 and Rab125 and their combination could inhibit the pulldown of Rab12 by GST-RILP, supporting the idea that peptides could influence the binding efficacy of Rab12 to its effectors and also that inhibition of both binding sites may be required (FIG. 17). Similar experiments will be performed to test the efficacy of the peptides and their combinations on Rab12 interactions with RILP, RILP-L1 and RILP-L2 under basal conditions and conditions under which phosphorylation of Rab12 will be enhanced.

### Example 13

Functional Consequences of the Modulation of Rab12 Interaction with RILP in Intact PC12 Cells

To test the ability of peptide 125 to modulate Rab12-RILP interaction in an in vitro model for Parkinson's disease, PC12 cells were co-transfected with NPY-mRFP to label the cells SGs, and CA Rab12, the constitutively active mutant of Rab12 that preferably binds RILP, as indicated by its ability to induce perinuclear clustering of the SGs. The cells were then either left untreated, or incubated with rotenone for 48h. The latter pesticide, is a known inhibitor of mitochondrial complex I that is often used to recapitulate the biochemical lesions of PD. After 48h, cells were incubated for further 30 min with either vehicle or TAT-conjugated peptide 125, as indicated. Cells were fixed and immunostained for their primary cilia with antibodies directed against Ar113b and visualized by confocal microscopy. As expected, in the absence of TAT-125, the NPY-mRFP-labelled SGs clustered in a perinuclear region (FIG. 18). However, in cells that were incubated with the TAT-125 peptide, partial scattering of the SGs was detected (FIG. 18, black arrows), demonstrating the ability of the peptide to modulate the Rab12-RILP interaction also in intact cells. Furthermore, both cell morphology, which was distorted in the rotenone treated cells, as well as the primary cilia (FIG. 18, magenta arrows), regained their normal appearance and size following the treatment with TAT-125 (FIG. 18).

### Example 14

Differentiation Between RILP and RILP-L2 Interaction with pRab12

Inspection of the positioning of Serine 106 in the Rab12-RILP versus Rab12-RILP-L2 models reveals that Serine 106 in Rab12 is capped by arginines in RILP-L2, which are predicted to stabilize the negatively charged phophoserine residue and contribute to the protein-protein interaction of Rab12 with RILP-L2. In contrast, Glutamic acid, E249, is positioned in the RILP interface, conveying a repulsive interaction when Serine 106 in Rab12 is phosphorylated (FIG. 19). Based on this model, peptides predicted to selectively inhibit phosphRab12 interaction with RILP-L2 while maintaining Rab12 interaction with RILP intact include peptides that share homology with the RILP-L2 derived sequence RPRPTLQELRD, including:

(SEQ ID NO: 9)
KPRHPENHLRK;
(SEQ ID NO: 10)
KPRHWEQTLRN;
(SEQ ID NO: 11)
KPRHWEQLLR;
(SEQ ID NO: 12)
LPRNMRQSLRI;
(SEQ ID NO: 13)
KPRHWEQTLRK;
(SEQ ID NO: 17)
KPRHKLQHLRK;
(SEQ ID NO: 18)
KPRHPEQHLRK;
(SEQ ID NO: 19)
KPRHPLQHLRK;
(SEQ ID NO: 20)
KPRHPEQTLRK;
(SEQ ID NO: 21)
KPRKDSQSLRF;
(SEQ ID NO: 22)
KPRHWEQLLRN;
(SEQ ID NO: 23)
KPRHKSTSLRD;
(SEQ ID NO: 24)
KPRKDLQSLRF;
(SEQ ID NO: 25)
LPRNARQNLRI;
(SEQ ID NO: 26)
HPRNHRQALRI;
(SEQ ID NO: 27)
HPRNMRQALRI;
(SEQ ID NO: 28)
LPRNARQSLRI;
(SEQ ID NO: 29)
HPRNMRQSLRI;
(SEQ ID NO: 30)
IPRNLRHNLRD;
(SEQ ID NO: 31)
LPRNARHELRS;
(SEQ ID NO: 32)
LPRNLRQNLRD;
and
(SEQ ID NO: 33)
VPRNLRHNLRD.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-32. (canceled)

33. A chimeric peptide comprising a sequence of 5 or more amino acids having at least 70% identity to SEQ ID NO:1 (ERFNSITSAYYR) derived from Interface 2 of the human Rab12 protein; and a sequence of 5 or more amino acids having at least 70% identity to SEQ ID NO: 2 (CKSTVGVDFKI) derived from Interface 1 of the human Rab12 protein.

34. The chimeric peptide of claim 33 comprising 5, 6, 7, 8, 9, 10, 11 or 12 amino acids derived from SEQ ID NO: 1 and 5, 6, 7, 8, 9, 10 or 11 amino acids derived from the SEQ ID NO:2.

35. The chimeric peptide of claim 33, wherein one or more serine (S) is replaced by another amino acid.

36. The chimeric peptide of claim 35, wherein the another amino acid is aspartate, glutamate, alanine, or Serine-phosphate.

37. The chimeric peptide of claim 33, wherein the Interface 2-derived sequence is selected from the group consisting of ERFNSITSAYYRSAK (peptide Rab121, SEQ ID NO: 4), ERFNDITSAYYRSAK (peptide Rab122, SEQ ID NO: 5), ERFNSITSAYYRDAK (peptide Rab123, SEQ ID NO: 6), and ERFNDITSAYYRDAK (peptide Rab124, SEQ ID NO: 7); and the Interface 1-derived sequence is EACKSTVGVDFKIKT (peptide Rab125, SEQ ID NO: 8).

38. The chimeric peptide of claim 33, having a linker between the Interface 2-derived sequence and the Interface 1-derived sequence.

39. The chimeric peptide of claim 38, wherein the linker has between 2-20 amino acids.

40. The chimeric peptide of claim 38, wherein the linker is a non-peptide linker.

41. The chimeric peptide of claim 33, wherein the chimeric peptide is linked to an internalization peptide or is lapidated or encapsulated thereby facilitating passage of the peptide across a cell membrane or a blood brain barrier.

42. A method of treating a subject suffering from a disease caused by imbalance of Rab12 phosphorylation or interactions with its effectors, comprising the step of administering to the subject an agent that inhibits the interaction of Rab12 with its effectors.

43. The method of claim 42, wherein the agent is a peptide comprising a sequence selected from the group consisting of: 5 or more amino acids of SEQ ID NO: 1, 5 or more amino acids of SEQ ID NO: 2, a combination of 5 or more amino acids of SEQ ID NO: 1 and 5 or more amino acids of SEQ ID NO: 2, SEQ ID NO: 3, SEQ ID NO: 9, SEQ ID NO: 10, SEQ ID NO: 11, SEQ ID NO: 12, SEQ ID NO: 13, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33.

44. The method of claim 42, wherein the agent is the chimeric peptide of claim 1.

45. The method of claim 42, wherein the effectors are RILP, RILP-like 1 (RILP-L1) or RILP-Like 2(RILP-L2).

46. The method of claim 42, wherein the disease is one or more of amyotrophic lateral sclerosis (ALS), Parkinson's disease, glaucoma, inflammatory disease, Crohn's disease, neurodegenerative disease, musician's dystonia (MD), writer's dystonia (WD), autism spectrum disorder, leprosy, or tuberculosis.

47. A peptide comprising an amino acid sequence selected from the group consisting of:
(SEQ ID NO: 3
RPRPTLQELRD;
(SEQ ID NO: 9)
KPRHPENHLRK;
(SEQ ID NO: 10)
KPRHWEQTLRN;
(SEQ ID NO: 11)
KPRHWEQLLR;
(SEQ ID NO: 12)
LPRNMRQSLRI;
(SEQ ID NO: 13)
KPRHWEQTLRK;
(SEQ ID NO: 17)
KPRHKLQHLRK;
(SEQ ID NO: 18)
KPRHPEQHLRK;
(SEQ ID NO: 19)
KPRHPLQHLRK;
(SEQ ID NO: 20)
KPRHPEQTLRK;
(SEQ ID NO: 21)
KPRKDSQSLRF;
(SEQ ID NO: 22)
KPRHWEQLLRN;
(SEQ ID NO: 23)
KPRHKSTSLRD;
(SEQ ID NO: 24)
KPRKDLQSLRF;
(SEQ ID NO: 25)
LPRNARQNLRI;
(SEQ ID NO: 26)
HPRNHRQALRI;
(SEQ ID NO: 27)
HPRNMRQALRI;
(SEQ ID NO: 28)
LPRNARQSLRI;
(SEQ ID NO: 29)
HPRNMRQSLRI;
(SEQ ID NO: 30)
IPRNLRHNLRD;
(SEQ ID NO: 31)
LPRNARHELRS;
(SEQ ID NO: 32)
LPRNLRQNLRD;
and
(SEQ ID NO: 33)
VPRNLRHNLRD,
wherein the peptide selectively inhibits phosphoRab12 interaction with RILP-L2 while maintaining Rab12 interaction with RILP intact.


SEQUENCE LISTING (33 sequences)

SEQ ID NO: 1 - protein, 12 aa, synthetic construct
  REGION 1..12: note = synthetic sequence
ERFNSITSAYYR

SEQ ID NO: 2 - protein, 11 aa, synthetic construct
  REGION 1..11: note = synthetic sequence
CKSTVGVDFKI

SEQ ID NO: 3 - protein, 11 aa, synthetic construct
  REGION 1..11: note = synthetic sequence
RPRPTLQELRD

SEQ ID NO: 4 - protein, 15 aa, synthetic construct
  REGION 1..15: note = synthetic sequence
ERFNSITSAYYRSAK

SEQ ID NO: 5 - protein, 15 aa, synthetic construct
  REGION 1..15: note = synthetic sequence
ERFNDITSAYYRSAK

SEQ ID NO: 6 - protein, 15 aa, synthetic construct
  REGION 1..15: note = synthetic sequence
ERFNSITSAYYRDAK

SEQ ID NO: 7 - protein, 15 aa, synthetic construct
  REGION 1..15: note = synthetic sequence
ERFNDITSAYYRDAK

SEQ ID NO: 8 - protein, 15 aa, synthetic construct
  REGION 1..15: note = synthetic sequence
EACKSTVGVDFKIKT

SEQ ID NO: 9 - protein, 11 aa, synthetic construct
  REGION 1..11: note = synthetic sequence
KPRHPENHLRK

SEQ ID NO: 10 - protein, 11 aa, synthetic construct
  REGION 1..11: note = synthetic sequence
KPRHWEQTLRN

SEQ ID NO: 11 - protein, 10 aa, synthetic construct
  REGION 1..10: note = synthetic sequence
KPRHWEQLLR

SEQ ID NO: 12 - protein, 11 aa, synthetic construct
  REGION 1..11: note = synthetic sequence
LPRNMRQSLRI

SEQ ID NO: 13 - protein, 11 aa, synthetic construct
  REGION 1..11: note = synthetic sequence
KPRHWEQTLRK

SEQ ID NO: 14 - protein, 28 aa, synthetic construct
  REGION 1..28: note = synthetic sequence
YGRKKRRQRRRGGEACKSTVGVDFKIKT

SEQ ID NO: 15 - DNA, 21 bp, synthetic construct
  misc_feature 1..21: note = synthetic sequence
  source 1..21: mol_type = other DNA
gaggcctgcaggtccaccgtg

SEQ ID NO: 16 - DNA, 18 bp, synthetic construct
  misc_feature 1..18: note = synthetic sequence
  source 1..18: mol_type = other DNA
gcagaacgtgtcgtcgtg

SEQ ID NO: 17 - protein, 11 aa, synthetic construct
  REGION 1..11: note = synthetic sequence
KPRHKLQHLRK

SEQ ID NO: 18 - protein, 11 aa, synthetic construct
  REGION 1..11: note = synthetic sequence
KPRHPEQHLRK

SEQ ID NO: 19 - protein, 11 aa, synthetic construct
  REGION 1..11: note = synthetic sequence
KPRHPLQHLRK

SEQ ID NO: 20 - protein, 11 aa, synthetic construct
  REGION 1..11: note = synthetic sequence
KPRHPEQTLRK

SEQ ID NO: 21 - protein, 11 aa, synthetic construct
  REGION 1..11: note = synthetic sequence
KPRKDSQSLRF

SEQ ID NO: 22 - protein, 11 aa, synthetic construct
  REGION 1..11: note = synthetic sequence
KPRHWEQLLRN

SEQ ID NO: 23 - protein, 11 aa, synthetic construct
  REGION 1..11: note = synthetic sequence
KPRHKSTSLRD

SEQ ID NO: 24 - protein, 11 aa, synthetic construct
  REGION 1..11: note = synthetic sequence
KPRKDLQSLRF

SEQ ID NO: 25 - protein, 11 aa, synthetic construct
  REGION 1..11: note = synthetic sequence
LPRNARQNLRI

SEQ ID NO: 26 - protein, 11 aa, synthetic construct
  REGION 1..11: note = synthetic sequence
HPRNHRQALRI

SEQ ID NO: 27 - protein, 11 aa, synthetic construct
  REGION 1..11: note = synthetic sequence
HPRNMRQALRI

SEQ ID NO: 28 - protein, 11 aa, synthetic construct
  REGION 1..11: note = synthetic sequence
LPRNARQSLRI

SEQ ID NO: 29 - protein, 11 aa, synthetic construct
  REGION 1..11: note = synthetic sequence
HPRNMRQSLRI

SEQ ID NO: 30 - protein, 11 aa, synthetic construct
  REGION 1..11: note = synthetic sequence
IPRNLRHNLRD

SEQ ID NO: 31 - protein, 11 aa, synthetic construct
  REGION 1..11: note = synthetic sequence
LPRNARHELRS

SEQ ID NO: 32 - protein, 11 aa, synthetic construct
  REGION 1..11: note = synthetic sequence
LPRNLRQNLRD

SEQ ID NO: 33 - protein, 11 aa, synthetic construct
  REGION 1..11: note = synthetic sequence
VPRNLRHNLRD